(19) United States
(12) Patent Application Publication
(10) Pub. No.: US 2008/0200890 A1
(43) Pub. Date: Aug. 21, 2008

(54) ANTIMICROBIAL DISPOSABLE ABSORBENT ARTICLES

(75) Inventors: Leigh E. Wood, Woodbury, MN (US); Alexis S. Statham, Woodbury, MN (US); Francis E. Porbeni, Woodbury, MN (US); Robert J. Maki, Hudson, WI (US); Jeremy M. Yarwood, Maplewood, MN (US); Matthew J. Schmid, Roberts, WI (US); Ronald W. Ausen, St. Paul, MN (US); Jay M. Jennen, Forest Lake, MN (US); Kelly S. Anderson, Houlton, WI (US); Matthew T. Scholz, Woodbury, MN (US); Robert W. Peterson, Spring Valley, WI (US); Erin A. Meulners, West St. Paul, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 12/098,517

(22) Filed: Apr. 7, 2008

Related U.S. Application Data
(63) Continuation-in-part of application No. 11/609,237, filed on Dec. 11, 2006.

(51) Int. Cl. A61F 13/53; A61F 13/20; B32B 27/18

(52) U.S. Cl. 604/360; 604/370; 604/385.17; 442/123

(57) ABSTRACT

Disposable absorbent articles comprising an absorbent material and a degradable thermoplastic polymer composition comprising an aliphatic polyester and an antimicrobial composition. The antimicrobial composition includes an antimicrobial component and an enhancer component. The aliphatic polyester and antimicrobial composition are formed into webs by melt extrusion, such as nonwovens and films, that are incorporated into disposable absorbent articles, such as disposable infant diapers, adult incontinence articles, feminine hygiene articles such as sanitary napkins, panty liners and tampons, personal care wipes and household wipes to provide odor control, control of microbial growth, and control of microbial toxin production.

### CROSS REFERENCE TO RELATED APPLICATIONS

This application is a continuation-in-part of U.S. application Ser. No. 11/609,237, filed Dec. 11, 2006, now pending, the disclosure of which is incorporated by reference in their entirety herein.

### TECHNICAL FIELD

The present invention relates to disposable absorbent articles formed from biodegradable aliphatic polyester polymers including antimicrobial compositions. These disposable absorbent articles are intended for absorbing body fluids, such as disposable infant diapers, feminine hygiene products including sanitary napkins, panty liners and tampons, products for adult incontinence, personal care wipes, and household wipes that include a microbial control material.

### BACKGROUND

A large variety of disposable absorbent articles are known in the art. These include personal absorbent articles used to absorb bodily fluids such as perspiration, urine, blood, and menses. Such articles also include disposable household wipes used to clean up similar fluids or typical household spills. These disposable absorbent articles are formed from thermoplastic polymers in the form of extruded films, foams, nonwovens or sometimes woven material. An issue with these articles is that they are designed for short term use but may not be disposed of immediately so that there is an opportunity for microorganisms to grow prior to disposal creating issues with formation of toxins, irritants or odor. However these absorbent articles are eventually disposed of so that the ability to form these absorbent articles of degradable thermoplastic materials is highly desirable.

One type of disposable absorbent articles is disposable absorbent garments such as infant diapers or training pants, products for adult incontinence, feminine hygiene products such as sanitary napkins and panty liners and other such products as are well known in the art. The typical disposable absorbent garment of this type is formed as a composite structure including an absorbent assembly disposed between a liquid permeable bodyside liner and a liquid impermeable outer cover. These components can be combined with other materials and features such as elastic materials and containment structures to form a product that is specifically suited to its intended purposes. Feminine hygiene tampons are also well known and generally are constructed of an absorbent assembly and sometimes an outer wrap of a fluid pervious material. Personal care wipes and household wipes are well known and generally include a substrate material, which may be a woven, knitted, or nonwoven material, and often contain functional agents such as cleansing solutions and the like.

An issue with these articles is that once body fluids, or household spills, are absorbed into the articles various microbes can grow in these articles. A well known problem with such articles is the generation of malodors associated with microbial growth and metabolites. For disposable absorbent articles such as infant diapers, products for adult incontinence, and feminine hygiene products the generation of such malodors can be a source of embarrassment for the user of these products. This can be particularly true for users of adult incontinence and feminine hygiene products. The issue of generation of malodor can include odors that are potentially detectable while the article is being worn and additionally after the article is disposed. In the case of household wipes the microbe associated generation of malodor is undesirable and can be embarrassing. Additionally the growth of bacteria and other microbes in such household wipes may lead to the undesired spreading of such microbes if the wipe is used subsequent to such microbial growth.

Various odor control solutions include masking, i.e., covering the odor with a perfume, absorbing the odor already present in the bodily fluids and those generated after degradation, or preventing the formation of odors that are associated with microbial growth. Examples of approaches to controlling the generation of malodor by controlling microbial growth include U.S. Pat. No. 6,767,508, which teaches the use of nonwoven fabrics that have been treated with an alkyl polyglycoside surfactant solution to result in a heterogeneous system having antibacterial activity when in contact with an aqueous source of bacteria. As discussed in U.S. Pat. No. 6,855,134 the dominant offensive malodors arising from urine biotransformation and urine decomposition are sulfurous compounds and ammonia.

An additional problem that is known to be associated with the use of some disposable absorbent articles, such as tampons, is that of specific bacteria producing harmful toxins. For example, toxic shock syndrome toxin (TSST) produced by Stapylococcus aureus can cause toxic shock syndrome (TSS) in non-immune humans. An increased incidence of TSS is associated with growth of S. aureus in the presence of tampons, such as those used in nasal packing or as catamenial devices. There is a need to provide a product that is effective at reducing these toxins that is also easily manufactured and preferably degradable following use.

The use of biodegradable polymers has been described to reduce the amount of waste materials land-filled and the number of disposal sites. Biodegradable materials have adequate properties to permit them to break down when exposed to conditions which lead to composting. Examples of materials thought to be biodegradable include aliphatic polyesters such as poly(lactic acid), poly(glycolic acid), poly(caprolactone), copolymers of lactide and glycolide, poly(ethylene succinate), and combinations thereof.

Degradation of aliphatic polyesters can occur through multiple mechanisms including hydrolysis, transesterification, chain scission, and the like. Instability of such polymers during processing can occur at elevated temperatures as described in WO 94/07941 (Gruber et. al.).

The processing of aliphatic polyesters as microfibers has been described in U.S. Pat. No. 6,645,618. U.S. Pat. No. 6,111,160 (Gruber et. al.) discloses the use of melt stable polylactides to form nonwoven articles via melt blown and spunbound processes.

Antimicrobial polymer compositions are known, as exemplified by U.S. Pat. Nos. 5,639,466 (Ford et. al.) and 6,756,428 (Denesuk). The addition of antimicrobial agents to hydrophilic polypropylene fibers having antimicrobial activity has been described in U.S. Patent Application Publication No. 2004/0241216 (Klun et. al.). These fibrous materials include nonwovens, wovens, knit webs, and knit batts.

The synergistic effect of antimicrobial agents, such as fatty acid monoesters, and enhancers have been described in WO 00/71183 (Andrews et. al.) and U.S. Patent Application Publication 2005/0089539 (Scholz et. al.) both herein incorporated by reference.

### BRIEF DESCRIPTION OF THE DRAWINGS

FIG. 1 illustrates a line graph of antimicrobial activity of Examples 10, 11 and 13 against S. aureus.

FIG. 2 illustrates a bar graph of antimicrobial activity of Examples 9-13 against high numbers of Proteus mirabilis in the presence of artificial urine.

FIG. 3 illustrates a bar graph of antimicrobial activity of Examples 11 and 13 against low numbers of P. mirabilis in the presence of artificial urine.

FIG. 4 illustrates a bar graph of viable P. mirabilis recovered after odor testing of Examples 11-13 in the presence of artificial urine.

FIG. 5 illustrates a bar graph of TSST production by S. aureus in the presence of extracts from Examples 9, 11 and 12.

FIG. 6 illustrates a bar graph of TSST production by S. aureus in Example 12 compared to that in a standard tampon.

### DISCLOSURE OF INVENTION

The present disclosure is directed to disposable absorbent articles formed with a degradable thermoplastic aliphatic polyester including an antimicrobial (preferably biocompatible) composition, which are preferably dry prior to use. The antimicrobial compositions, or components thereof, are used as melt additives in the melt-processable degradable thermoplastic aliphatic polyester polymer and includes an antimicrobial component and an enhancer. The melt-processable degradable aliphatic polyester with the included antimicrobial component and enhancer can be easily and directly formed into disposable absorbent articles without additional coating or loading steps greatly simplifying the manufacture of these disposable absorbent articles. The melt processed antimicrobial component and enhancer are stable prior to both the manufacture of the final disposable absorbent article and the ultimate end use providing extended antimicrobial activity. Further, when exposed to moisture when ultimately used the degradable aliphatic polyester at least partially degrades or hydrolyzes assisting in releasing the antimicrobial composition or component into the surrounding environment.

For disposable absorbent articles of the present invention, that are disposable absorbent garments of the type that are composite structures including an absorbent assembly disposed between a liquid permeable bodyside liner and a liquid impermeable outer cover, the degradable thermoplastic aliphatic polyester polymer including an antimicrobial composition can preferably be in the form of a nonwoven material or loose fibers that are positioned within the absorbent assembly (e.g. distributed within the bulk of the absorbent), on the body facing side of the absorbent, or on the opposite side of the absorbent assembly. Alternately the degradable thermoplastic aliphatic polyester polymer including an antimicrobial composition can be formed into the liquid permeable bodyside liner. Alternately the degradable thermoplastic aliphatic polyester polymer including an antimicrobial composition can be formed into a film that can be positioned on the liquid impermeable outer cover side of the absorbent assembly, or the film can serve as the liquid impermeable outer cover of the disposable absorbent garment.

When the disposable absorbent article of the present invention is a tampon the degradable thermoplastic aliphatic polyester polymer including an antimicrobial composition can be in the form of a nonwoven material or loose fibers that are positioned within the absorbent assembly or, when a nonwoven, it can serve as the fluid pervious outer wrap of the tampon.

When the disposable absorbent articles of the present invention are a personal care or household wipe the substrate of the wipe can be made with, or incorporate, the aliphatic polyester with the included antimicrobial component and enhancer. For example the woven, knitted or nonwoven substrate can be made with a blend of fibers, one of which comprises the aliphatic polyester with the included antimicrobial component and enhancer. Generally the wipe would be formed from a nonwoven such as by carding or entanglement for one time or limited use applications. Alternatively aliphatic polyester fibers could be woven or knitted in whole or in part into a wipe product which could be used for longer periods. The inclusion of the antimicrobial component or composition into the degradable aliphatic polyester fibers gives the wipe extended antimicrobial activity over time. Additional fibers that could be blended in with the aliphatic polyesters include fibers to increase absorbency or other properties include fibers based on polyolefins, polyesters, acrylates, superabsorbent fibers, and natural fibers such as bamboo, soy bean, agave, coco, rayon, cellulosics, wood pulp or cotton.

Nonwoven webs of the aliphatic polyester with the included antimicrobial component and enhancer can be prepared via any standard process for directly making nonwoven webs, including spunbond, blown microfiber and nanofiber processes. Additionally fibers or filaments can be prepared with the aliphatic polyester with the included antimicrobial component and enhancer and such fibers or filaments can be cut to desired lengths and further processed into nonwoven webs using various known web forming processes, such as carding. In such cases the chopped fibers may be blended with other fibers in the web forming process. Alternatively fibers or filaments prepared with the aliphatic polyester with the included antimicrobial component and enhancer could be woven or knitted alone or in combination with other fibers.

In one aspect, the disposable absorbent article includes a melt formed aliphatic polyester composition comprising a thermoplastic aliphatic polyester; an antimicrobial component incorporated within the aliphatic polyester, in which the antimicrobial component is present at greater than 1 percent by weight of the aliphatic polyester; and an enhancer. The aliphatic polyester is in sufficient proportion to the antimicrobial component(s) with enhancers to yield an effective antimicrobial composition. The antimicrobial component(s) are selected from fatty acid esters of polyhydric alcohols, fatty ethers of polyhydric alcohols, hydroxy acid esters of fatty alcohols, alkoxylated derivatives thereof (having less than 5 moles of alkoxide group per mole of polyhydric alcohol) and combinations thereof. The enhancer provides for enhanced antimicrobial activity of the antimicrobial component(s) in the degradable aliphatic polyester composition.

Exemplary preferred aliphatic polyesters are poly(lactic acid), poly(glycolic acid), poly(lactic-co-glycolic acid), blends, and copolymers thereof. The antimicrobial component may be selected from (C₇-C₁₄) saturated fatty acid esters of a polyhydric alcohol or (C₈-C₂₂) unsaturated fatty acid esters of a polyhydric alcohol such as propylene glycol monoesters and glycerol monoesters. Examples are propylene glycol monolaurate, propylene glycol monocaprylate, glycerol monolaurate, and combinations thereof.

Inventive disposable absorbent articles include disposable diapers, adult incontinent articles or pads, feminine pads, sanitary napkins, catamenial tampons, dental tampons, medical tampons, surgical tampons, nasal tampons or wipes (such as personal cleansing or household wipes) that are preferably dry prior to use but are moist or wet in their end use environment. These disposable absorbent articles are formed using polymeric sheets, polymeric fibers, woven webs, knitted webs, nonwoven webs, porous membranes, polymeric foams, thermal or adhesive laminates, layered compositions, and combinations thereof made of the degradable aliphatic polyester polymer including an antimicrobial composition as described above.

Desirably, antimicrobial components of the antimicrobial composition when wet are released into the surrounding medium in which microbes are to be controlled. The antimicrobial components are released as the aliphatic polyester degrades and/or swells when wet, giving the aliphatic polyester, in some measure, a self-disinfecting property. The degradation of the aliphatic polyester may be controlled to some extent to adjust the release characteristics of the antimicrobial component when exposed to moisture. The antimicrobial properties of the degradable aliphatic polyester polymer with the antimicrobial component(s) and enhancer also potentially delays the degradation of the degradable aliphatic polyester polymer or the disposable absorbent article until after use. Prior to use the degradable aliphatic polyester polymer composition is generally dry and the antimicrobial composition or component is in a generally stable form within the degradable aliphatic polyester polymer matrix.

### DETAILED DESCRIPTION OF INVENTION

For the following defined terms, these definitions shall be applied, unless a different definition is given in the claims or elsewhere in the specification.

The term “antimicrobial” or “antimicrobial activity” means having sufficient antimicrobial activity to kill pathogenic and non-pathogenic microorganisms including bacteria, fungi, algae and virus, prevent the growth/reproduction of pathogenic and non-pathogenic microorganisms or control the production of exoproteins, such as toxic shock syndrome toxin (TSST).

The term “biodegradable” or “degradable” means degradable by the action of naturally occurring microorganisms such as bacteria, fungi and algae and/or natural environmental factors such as hydrolysis, transesterification, exposure to ultraviolet or visible light (photodegradable) and enzymatic mechanisms or combinations thereof.

The term “biocompatible” means biologically compatible by not producing toxic, injurious or immunological responses in living tissue. Biocompatible materials may also be broken down by biochemical and/or hydrolytic processes and absorbed by living tissue.

The term “sufficient amount” or “effective amount” means the amount of the antimicrobial component and/or enhancer when in a composition, as a whole, provides an antimicrobial (including, for example, antiviral, antibacterial, or antifungal) activity that reduces, prevents growth of, or eliminates colony forming units for one or more species of microorganisms such that an acceptable level of the organism results.

The term “enhancer” means a component that enhances the effectiveness of the antimicrobial component such that when the composition without the enhancer is used separately, it does not provide the same level of antimicrobial activity as the composition including enhancer. The enhancement may be in speed of antimicrobial activity, extent of antimicrobial activity, greater spectrum of activity or combinations thereof. An enhancer in the absence of the antimicrobial component may not provide any appreciable antimicrobial activity. The enhancing effect may also not be seen for all microorganisms.

The term “fatty” means a straight or branched chain alkyl or alkylene moiety having 6 to 22 (odd or even number) carbon atoms, unless otherwise specified.

The recitation of numerical ranges by endpoints includes all numbers subsumed within that range.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Aliphatic polyesters useful in the present invention include homo- and copolymers of poly(hydroxyalkanoates) and homo- and copolymers of those aliphatic polyesters derived from the reaction product of one or more polyols with one or more polycarboxylic acids and is typically formed from the reaction product of one or more alkanediols with one or more alkanedicarboxylic acids (or acyl derivatives). Aliphatic polyesters may further be derived from multifunctional polyols, e.g. glycerin, sorbitol, pentaerythritol, and combinations thereof, to form branched, star, and graft homo- and copolymers. Miscible and immiscible blends of aliphatic polyesters with one or more additional semicrystalline or amorphous polymers may also be used.

One useful class of aliphatic polyesters are poly(hydroxyalkanoates), derived by condensation or ring-opening polymerization of hydroxy acids, or derivatives thereof. Suitable poly(hydroxyalkanoates) may be represented by the formula:

H(O—R—C(O)—)ₙOH,

where R is an alkylene moiety that may be linear or branched having 1 to 20 carbon atoms, preferably 1 to 12 carbon atoms optionally substituted by catenary (bonded to carbon atoms in a carbon chain) oxygen atoms; n is a number such that the ester is polymeric, and is preferably a number such that the molecular weight of the aliphatic polyester is at least 10,000, preferably at least 30,000, and most preferably at least 50,000 daltons. Although higher molecular weight aliphatic polyester polymers generally yield films with better mechanical properties. It is a significant advantage of the present invention that the antimicrobial component in many embodiments plasticizes the aliphatic polyester component allowing for melt processing of higher molecular weight aliphatic polyester polymers. Thus, the molecular weight of the aliphatic polyester is typically less than 1,000,000, preferably less than 500,000, and most preferably less than 300,000 daltons. R may further comprise one or more caternary (i.e. in chain) ether oxygen atoms. Generally, the R group of the hydroxy acid is such that the pendant hydroxyl group is a primary or secondary hydroxyl group.

Useful poly(hydroxyalkanoates) include, for example, homo- and copolymers of poly(3-hydroxybutyrate), poly(4-hydroxybutyrate), poly(3-hydroxyvalerate), poly(lactic acid) (also known as polylactide), poly(3-hydroxypropanoate), poly(4-hydropentanoate), poly(3-hydroxypentanoate), poly(3-hydroxyhexanoate), poly(3-hydroxyheptanoate), poly(3-hydroxyoctanoate), polydioxanone, polycaprolactone, and polyglycolic acid (i.e. polyglycolide). Copolymers of two or more of the above hydroxy acids may also be used, for example, poly(3-hydroxybutyrate-co-3-hydroxyvalerate), poly(lactate-co-3-hydroxypropanoate), poly(glycolide-co-p-dioxanone), and poly(lactic acid-co-glycolic acid). Blends of two or more of the poly(hydroxyalkanoates) may also be used, as well as blends with one or more semicrystalline or amorphous polymers and/or copolymers.

The aliphatic polyester may be a block copolymer of poly(lactic acid-co-glycolic acid). Aliphatic polyesters useful in the degradable aliphatic polyester polymer compositions may include homopolymers, random copolymers, block copolymers, star-branched random copolymers, star-branched block copolymers, dendritic copolymers, hyperbranched copolymers, graft copolymers, and combinations thereof.

Another useful class of aliphatic polyesters includes those aliphatic polyesters derived from the reaction product of one or more alkane diols with one or more alkanedicarboxylic acids (or acyl derivatives). Such aliphatic polyesters have the general formula:

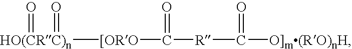

where R′ and R″ each represent an alkylene moiety that may be linear or branched having from 1 to 20 carbon atoms, preferably 1 to 12 carbon atoms, and m is a number such that the ester is polymeric, and is preferably a number such that the molecular weight of the aliphatic polyester is at least 10,000, preferably at least 30,000, and most preferably at least 50,000 daltons, but less than 1,000,000, preferably less than 500,000 and most preferably less than 300,000 daltons. Each n is independently 0 or 1, R′ and R″ may further comprise one or more caternary (i.e. in chain) ether oxygen atoms.

Examples of aliphatic polyesters include those homo- and copolymers derived from (a) one or more of the following diacids (or derivative thereof): succinic acid, adipic acid, 1,12 dicarboxydodecane, fumaric acid, glutartic acid, diglycolic acid, and maleic acid; and (b) one of more of the following diols: ethylene glycol, polyethylene glycol, propanediols, butanediols, hexanediol, alkane diols having 5 to 12 carbon atoms, diethylene glycol, polyethylene glycols having a molecular weight of 300 to 10,000 daltons, preferably 400 to 8,000 daltons, propylene glycols having a molecular weight of 300 to 4000 daltons, block or random copolymers derived from ethylene oxide, propylene oxide, or butylene oxide, dipropylene glycol and polypropylene glycol, and (c) optionally a small amount, i.e. 0.5-7.0 mole % of a polyol with a functionality greater than two such as glycerol, neopentyl glycol, and pentaerythritol.

Such polymers may include polybutylenesuccinate homopolymer, polybutylene adipate homopolymer, polybutyleneadipate-succinate copolymer, polyethylenesuccinate-adipate copolymer, polyethylene glycol succinate and polyethylene adipate homopolymer.

Commercially available aliphatic polyesters include poly(lactide), poly(glycolide), poly(lactide-co-glycolide), poly(L-lactide-co-trimethylene carbonate), poly(dioxanone), poly(butylene succinate), and poly(butylene adipate).

Useful aliphatic polyesters include those derived from semicrystalline polylactic acid. Poly(lactic acid) or polylactide has lactic acid as its principle degradation product. The aliphatic polyester polymer may be prepared by ring-opening polymerization of the lactic acid dimer, lactide. Lactic acid is optically active and the dimer appears in four different forms: L,L-lactide, D,D-lactide, D,L-lactide (meso lactide) and a racemic mixture of L,L- and D,D-.

The polylactide preferably has a high enantiomeric ratio to maximize the intrinsic crystallinity of the aliphatic polyester polymer. The degree of crystallinity of a poly(lactic acid) is based on the regularity of the aliphatic polyester polymer backbone and the ability to crystallize with other aliphatic polyester polymer chains. If relatively small amounts of one enantiomer (such as D-) is copolymerized with the opposite enantiomer (such as L-) the aliphatic polyester polymer chain becomes irregularly shaped, and becomes less crystalline. If crystallinity is favored, it is desirable to have a poly(lactic acid) that is at least 85% of one isomer, at least 90%, or at least 95% in order to maximize the crystallinity.

An approximately equimolar blend of D-polylactide and L-polylactide is also useful. This blend forms a unique crystal structure having a higher melting point (˜210° C.) than does either the D-poly(lactide) and L-(polylactide) alone (˜190° C.), and has improved thermal stability, see H. Tsuji et. al., Polymer, 40 (1999) 6699-6708.

Copolymers, including block and random copolymers, of poly(lactic acid) with other aliphatic polyesters may also be used. Useful co-monomers include glycolide, beta-propiolactone, tetramethylglycolide, beta-butyrolactone, gamma-butyrolactone, pivalolactone, 2-hydroxybutyric acid, alpha-hydroxyisobutyric acid, alpha-hydroxyvaleric acid, alpha-hydroxyisovaleric acid, alpha-hydroxycaproic acid, alpha-hydroxyethylbutyric acid, alpha-hydroxyisocaproic acid, alpha-hydroxy-beta-methylvaleric acid, alpha-hydroxyoctanoic acid, alpha-hydroxydecanoic acid, alpha-hydroxymyristic acid, and alpha-hydroxystearic acid.

Blends of poly(lactic acid) and one or more other aliphatic polyesters, or one or more other polymers may also be used. Examples of useful blends include poly(lactic acid) and poly(vinyl alcohol), polyethylene glycol/polysuccinate, polyethylene oxide, polycaprolactone and polyglycolide.

The molecular weight of the degradable aliphatic polyester polymer should be chosen so that the aliphatic polyester polymer may be processed as a melt. For polylactide, for example, the molecular weight may be from about 10,000 to 1,000,000 daltons, and is preferably from about 30,000 to 300,000 daltons. By “melt-processable” it is meant that the degradable aliphatic polyesters are fluid or can be pumped or extruded at the temperatures used to process the articles (e.g. fibers, nonwovens or films) and do not degrade or gel at those temperatures to the extent that the physical properties are unusable for the intended disposable absorbent article. Materials used to form the invention absorbent disposable articles may be made into films by extrusion, casting, thermal pressing, and the like. The materials used to form the invention disposable absorbent articles can be made into fibers or nonwovens using melt processes such as spun bond, blown microfiber, melt spinning and the like. Certain embodiments also may be injection molded. Generally, weight average molecular weight (M_{w}) of the aliphatic polyester polymers is above the entanglement molecular weight, as determined by a log-log plot of viscosity versus number average molecular weight (Mₙ). Above the entanglement molecular weight, the slope of the plot is about 3.4, whereas the slope of lower molecular weight aliphatic polyester polymers is 1.

The aliphatic polyester typically comprises at least 50 weight percent, preferably at least 60 weight percent, and most preferably at least 65 weight percent of the degradable aliphatic polyester polymer compositions.

For melt processing, preferred antimicrobial components have low volatility and do not decompose appreciably under melt process conditions. The preferred antimicrobial components contain less than 2 wt. % water, and more preferably less than 0.10 wt. % (determined by Karl Fischer analysis).

The antimicrobial component content in the degradable aliphatic polyester polymer composition (as it is ready-to-use) is typically at least 1 wt. %, 2 wt. %, 5 wt. %, 10 wt. % and sometimes greater than 15 wt. %. In certain embodiments, in which a low tensile strength is desired or acceptable, the antimicrobial component comprises greater than 20 wt. %, greater than 25 wt. %, or even greater than 30 wt. % of the degradable aliphatic polyester polymer composition.

The antimicrobial component may include one or more fatty acid esters of a polyhydric alcohol, fatty ethers of a polyhydric alcohol, or alkoxylated derivatives thereof (of either or both of the ester and/or ether), or combinations thereof. More specifically, the antimicrobial component is selected from the group consisting of a (C₇-C₁₄) saturated fatty acid ester of a polyhydric alcohol (preferably, a (C₈-C₁₂) saturated fatty acid ester of a polyhydric alcohol), an (C₇-C₂₂) unsaturated fatty acid ester of a polyhydric alcohol (preferably, an (C₈-C₁₈) unsaturated fatty acid ester of a polyhydric alcohol), a (C₇-C₂₂) saturated fatty ether of a polyhydric alcohol (preferably, a (C₇-C₁₈) saturated fatty ether of a polyhydric alcohol), an (C₇-C₂₂) unsaturated fatty ether of a polyhydric alcohol (preferably, an (C₈-C₁₈) unsaturated fatty ether of a polyhydric alcohol), an alkoxylated derivative thereof, and combinations thereof. Preferably, the esters and ethers are monoesters and monoethers, unless they are esters and ethers of sucrose in which case they can be monoesters, diesters, monoethers, or diethers. Various combinations of monoesters, diesters, monoethers, and diethers can be used in a composition of the present invention.

Preferably the (C₇-C₁₄) saturated and (C₇-C₂₂) unsaturated monoesters and monoethers of polyhydric alcohols are at least 80% pure (having 20% or less diester and/or triester or diether and/or triether), more preferably 85% pure, even more preferably 90% pure, most preferably 95% pure. Impure esters or ethers would not have sufficient, if any, antimicrobial activity.

Useful fatty acid esters of a polyhydric alcohol may have the formula:

(R¹—C(O)—O)ₙ—R²

wherein R¹ is the residue of a (C₇-C₁₄) saturated fatty acid (preferably, a (C₈-C₁₂) saturated fatty acid), or a (C₇-C₂₂) unsaturated (preferably, a C₈-C₁₈) unsaturated, including polyunsaturated) fatty acid, R² is the residue of a polyhydric alcohol (typically and preferably, glycerin, propylene glycol, and sucrose, although a wide variety of others can be used including pentaerythritol, sorbitol, mannitol, xylitol, etc.), and n=1 or 2. The R² group includes at least one free hydroxyl group (preferably, residues of glycerin, propylene glycol, or sucrose). Preferred fatty acid esters of polyhydric alcohols are esters derived from C₈, C₉, C₁₀, C₁₁, and C₁₂ saturated fatty acids. For embodiments in which the polyhydric alcohol is glycerin or propylene glycol, n=1, although when it is sucrose, n=1 or 2. In general, monoglycerides derived from C₁₀ to C₁₂ fatty acids are food grade materials and GRAS materials.

Fatty acid monoesters, such as glycerol monoesters of lauric, caprylic, capric, and heptanoic acid and/or propylene glycol monoesters of lauric, caprylic, capric and heptanoic acid, are active against Gram-positive bacteria, fungi, yeasts and lipid coated viruses but alone are not generally as effective against Gram-negative bacteria. When the fatty acid monoesters are combined with the enhancers described below, the composition can have greater efficacy against Gram-negative bacteria.

Exemplary fatty acid monoesters include, but are not limited to, glycerol monoesters of lauric (monolaurin), caprylic (monocaprylin), and capric (monocaprin) acid, and propylene glycol monoesters of lauric, caprylic, and capric acid, as well as lauric, caprylic, and capric acid monoesters of sucrose. Other fatty acid monoesters include glycerin and propylene glycol monoesters of oleic (18:1), linoleic (18:2), linolenic (18:3), and arachonic (20:4) unsaturated (including polyunsaturated) fatty acids. 18:1, for example, means the compound has 18 carbon atoms and 1 carbon-carbon double bond. Preferred unsaturated chains have at least one unsaturated group in the cis isomer form. In certain preferred embodiments, the fatty acid monoesters that are suitable for use in the present composition include known monoesters of lauric, caprylic, and capric acid, such as that known as GML or the trade designation LAURICIDIN (the glycerol monoester of lauric acid commonly referred to as monolaurin or glycerol monolaurate), glycerol monocaprate, glycerol monocaprylate, propylene glycol monolaurate, propylene glycol monocaprate, propylene glycol monocaprylate, and combinations thereof.

Exemplary fatty acid diesters of sucrose include, but are not limited to, lauric, caprylic, and capric diesters of sucrose as well as combinations thereof.

A fatty ether of a polyhydric alcohol is preferably of the formula:

(R—O)ₙ—R⁴,

wherein R³ is a (C₇-C₁₄) saturated aliphatic group (preferably, a (C₈-C₁₂) saturated aliphatic group), or a (C₇-C₂₂) unsaturated (preferably, (C₈-C₁₈) unsaturated, including polyunsaturated) aliphatic group, R⁴ is the residue of a polyhydric alcohol. Preferred polyhydric alcohols include glycerin, sucrose, or propylene glycol. For glycerin and propylene glycol n=1, and for sucrose n=1 or 2. Preferred fatty ethers are monoethers of (C₇-C₁₄) alkyl groups (more preferably, (C₈-C₁₂) alkyl groups).

Exemplary fatty monoethers include, but are not limited to, laurylglyceryl ether, caprylglycerylether, caprylylglyceryl ether, laurylpropylene glycol ether, caprylpropyleneglycol ether, and caprylylpropyleneglycol ether. Other fatty monoethers include glycerin and propylene glycol monoethers of oleyl (18:1), linoleyl (18:2), linolenyl (18:3), and arachonyl (20:4) unsaturated and polyunsaturated fatty alcohols. In certain preferred embodiments, the fatty monoethers that are suitable for use in the present composition include laurylglyceryl ether, caprylglycerylether, caprylyl glyceryl ether, laurylpropylene glycol ether, caprylpropyleneglycol ether, caprylylpropyleneglycol ether, and combinations thereof. Unsaturated chains preferably have at least one unsaturated bond in the cis isomer form.

The alkoxylated derivatives of the aforementioned fatty acid esters and fatty ethers (e.g., one which is ethoxylated and/or propoxylated on the remaining alcohol groups) also have antimicrobial activity as long as the total alkoxylate is kept relatively low. Preferred alkoxylation levels are disclosed in U.S. Pat. No. 5,208,257. If the esters and ethers are ethoxylated, total moles of ethylene oxide are preferably less than 5, more preferably less than 2. The fatty acid esters or fatty ethers of polyhydric alcohols can be alkoxylated, preferably ethoxylated and/or propoxylated, by conventional techniques. Alkoxylating compounds are preferably selected from the group consisting of ethylene oxide, propylene oxide, and mixtures thereof, and similar oxirane compounds.

The degradable aliphatic polyester polymer compositions typically include a total amount of fatty acid esters, fatty ethers, alkoxylated fatty acid esters, or alkoxylated fatty ethers of at least 1 weight percent (wt. %), at least 2 wt. %, greater than 5 wt. %, at least 6 wt. %, at least 7 wt. %, at least 10 wt. %, at least 15 wt. %, or at least 20 wt. %, based on the total weight of the ready-to-use composition or the degradable thermoplastic aliphatic polyester composition. The term “ready-to-use” means the composition in its intended form for use and is generally the degradable thermoplastic aliphatic polyester composition. In a preferred embodiment, they are present in a total amount of no greater than 60 wt. %, no greater than 50 wt. %, no greater than 40 wt. %, or no greater than 35 wt. %, based on the total weight of the ready-to-use composition. Alternatively, these proportions may be considered relative to the aliphatic polyester (based on 100 parts by weight of the aliphatic polyester), i.e., no greater than 150 parts fatty acid ester, 100 parts fatty acid ester, 67 parts fatty acid ester and 54 parts fatty acid ester. Certain compositions may be higher in concentration if they are intended to be used as a “masterbatch” for additional processing. As used herein, the term, “masterbatch” refers to a concentrate that is added to a composition that is melt processed

Degradable aliphatic polyester polymer compositions or antimicrobial compositions of the present invention that include one or more fatty acid monoesters, fatty monoethers, hydroxyl acid esters of alcohols or alkoxylated derivatives thereof can also include a small amount of a di- or tri-fatty acid ester (i.e., a fatty acid di- or tri-ester), a di- or tri-fatty ether (i.e., a fatty di- or tri-ether), or alkoxylated derivative thereof. Preferably, such components comprise no more than 10 wt. %, no more than 7 wt. %, no more than 6 wt. %, or no more than 5 wt. %, of the total weight of the antimicrobial component to preserve the antimicrobial efficacy of the antimicrobial component as discussed above.

An additional class of antimicrobial component is a fatty alcohol ester of a hydroxyl functional carboxylic acid preferably of the formula:

R⁵—O—(—C(O)—R⁶—O)ₙH,

wherein R⁵ is the residue of a (C₇-C₁₄) saturated alkyl alcohol (preferably a (C₈-C₁₂) saturated alkyl alcohol) or a (C₈-C₂₂) unsaturated alcohol (including polyunsaturated alcohol), R⁶ is the residue of a hydroxycarboxylic acid wherein the hydroxycarboxylic acid has the following formula:

R⁷(CR⁸OH)ₚ(CH₂)_{q}COOH,

wherein: R⁷ and R⁸ are each independently H or a (C₁-C₈) saturated straight, branched, or cyclic alkyl group, a (C₆-C₁₂) aryl group, or a (C₆-C₁₂) aralkyl or alkaryl group wherein the alkyl groups are saturated straight, branched, or cyclic, wherein R⁷ and R⁸ may be optionally substituted with one or more carboxylic acid groups; p=1 or 2; and q=0-3; and n=1, 2, or 3. The R⁶ group may include one or more free hydroxyl groups but preferably is free of hydroxyl groups. Preferred fatty alcohol esters of hydroxycarboxylic acids are esters derived from branched or straight chain C₈, C₉, C₁₀, C₁₁, or C₁₂ alkyl alcohols. The hydroxyacids typically have one hydroxyl group and one carboxylic acid group.

In one aspect, the antimicrobial component includes a (C₇-C₁₄, preferably C₈-C₁₂) saturated fatty alcohol monoester of a (C₂-C₈) hydroxycarboxylic acid, a (C₈-C₂₂) mono- or poly-unsaturated fatty alcohol monoester of a (C₂-C₈) hydroxycarboxylic acid, an alkoxylated derivative of either of the foregoing, or combinations thereof. The hydroxycarboxylic acid moiety can include aliphatic and/or aromatic groups. For example, fatty alcohol esters of salicylic acid are possible. As used herein, a “fatty alcohol” is an alkyl or alkylene monofunctional alcohol having an even or odd number of carbon atoms.

Exemplary fatty alcohol monoesters of hydroxycarboxylic acids include, but are not limited to, (C₈-C₁₂) fatty alcohol esters of lactic acid such as octyl lactate, 2-ethylhexyl lactate (Purasolv EHL from Purac, Lincolnshire Ill., lauryl lactate (Chrystaphyl 98 from Chemic Laboratories, Canton Mass.), lauryl lactyl lacate, 2-ethylhexyl lactyl lactate; (C₈-C₁₂) fatty alcohol esters of glycolic acid, lactic acid, 3-hydroxybutanoic acid, mandelic acid, gluconic acid, tartaric acid, and salicylic acid.

The alkoxylated derivatives of the fatty alcohol esters of hydroxy functional carboxylic acids (e.g., one which is ethoxylated and/or propoxylated on the remaining alcohol groups) also have antimicrobial activity as long as the total alkoxylate is kept relatively low. The preferred alkoxylation level is less than 5 moles, and more preferably less than 2 moles, per mole of hydroxycarboxylic acid.

The above antimicrobial components comprising an ester linkage are hydrolytically sensitive, and may be degraded by exposure to water, particularly at extreme pH levels (less than 4 or more than 10) or by certain bacteria that can enzymatically hydrolyze the ester to the corresponding acid and alcohol, which may be desirable in certain applications. For example, an article may be made to degrade rapidly by incorporating an antimicrobial component comprising at least one ester group. If extended persistence of the disposable article is desired such as for a multiple use household wipe, an antimicrobial component, free of hydrolytically sensitive groups, may be used. For example, the fatty monoethers are not hydrolytically sensitive under ordinary processing conditions, and are resistant to microbial attack.

An optional additional component that can be included in the antimicrobial composition of the degradable aliphatic polyester polymer including an antimicrobial composition includes cationic amine antimicrobial compounds, which include antimicrobial protonated tertiary amines and small molecule quaternary ammonium compounds.

Exemplary small molecule quaternary ammonium compounds include benzalkonium chloride and alkyl substituted derivatives thereof, di-long chain alkyl (C₈-C₁₈) quaternary ammonium compounds, cetylpyridinium halides and their derivatives, benzethonium chloride and its alkyl substituted derivatives, octenidine and compatible combinations thereof. Suitable small molecule quarternary ammonium compounds, typically comprise one or more quaternary ammonium group having attached thereto at least one C₆-C₁₈ linear or branched alkyl or aralkyl chain. Suitable compounds include those disclosed in Lea & Febiger, Chapter 13 in Block, S., Disinfection, Sterilization and Preservation, 4^{th} ed., 1991. Exemplary compounds within this class are: monoalkyltrimethylammonium salts, monoalkyldimethylbenzyl ammonium salts, dialkyldimethyl ammonium salts, benzethonium chloride, alkyl substituted benzethonium halides such as methylbenzethonium chloride and octenidine. Additional examples of quaternary ammonium antimicrobial components are: benzalkonium halides having an alkyl chain length of C₈-C₁₈, preferably C₁₂-C₁₆, more preferably a mixture of chain lengths, e.g., benzalkonium chloride comprising 40% C₁₂ alkyl chains, 50% C₁₄ alkyl chains, and 10% C₁₆ chains (available as Barquat MB-50 from Lonza Group Ltd.); benzalkonium halides substituted with alkyl groups on the phenyl ring (available as Barquat 4250); dimethyldialkylammonium halides having C₈-C₁₈ alkyl groups, or mixtures of such compounds (available as Bardac 2050, 205M and 2250 from Lonza); and cetylpyridinium halides such as cetylpyridinium chloride (Cepacol Chloride available as Cepacol Chloride from Merrell Labs); benzethonium halides and alkyl substituted benzethonium halides (available as Hyamine 1622 and Hyamine 10× from Rohm and Haas). Useful protonated tertiary amines have at least one C₆-C₁₈ alkyl group. When used the cationic antimicrobial components are typically added to the degradable aliphatic polyester polymer compositions at a concentration of at least 1.0 wt. %, preferably at least 3 wt. %, more preferably greater than 5.0 wt. %, still more preferably at least 6.0 wt. %, even more preferably at least 10 wt. % and most preferably at least 20.0 wt. %, in some cases exceeding 25 wt. %. Preferably, the concentration is less than 50 wt. %, more preferably less than 40 wt. %, and most preferably less than 35 wt. %. The cationic amine antimicrobial compounds can be added to the antimicrobial composition of the degradable aliphatic polyester polymer may be added to serve as preservatives and in some cases may enhance the antimicrobial activity of the degradable aliphatic polyester polymer including an antimicrobial composition.

The degradable aliphatic polyester polymer compositions include an enhancer (preferably a synergist) to enhance the antimicrobial activity especially against Gram-negative bacteria, e.g. Escherichia coli and Pseudomonas sp. The enhancer component may include an alpha-hydroxy acid, a beta-hydroxy acid, other carboxylic acids, a (C₂-C₆) saturated or unsaturated alkyl carboxylic acid, a (C₆-C₁₆) aryl carboxylic acid, a (C₆-C₁₆) aralkyl carboxylic acid, a (C₆-C₁₂) alkaryl carboxylic acid, a phenolic compound (such as certain antioxidants and parabens), a (C₅-C₁₀) monohydroxy alcohol, a chelating agent, a glycol ether (i.e., ether glycol), or oligomers that degrade to release one of the above enhancers. Examples of such oligomers are oligomers of glycolic acid, lactic acid or both having at least 4 or 6 repeat units. Various combinations of enhancers can be used if desired.

The alpha-hydroxy acid, beta-hydroxy acid, and other carboxylic acid enhancers are preferably present in their protonated, free acid form. It is not necessary for all of the acidic enhancers to be present in the free acid form; however, the preferred concentrations listed below refer to the amount present in the free acid form. Additional, non-alpha hydroxy acid, betahydroxy acid or other carboxylic acid enhancers, may be added in order to acidify the formulation or buffer it at a pH to maintain antimicrobial activity. Preferably, acids are used having a pKa greater than about 2.5, preferably greater than about 3, and most preferably greater than about 3.5 in order to avoid hydrolyzing the aliphatic polyester component. Furthermore, chelator enhancers that include carboxylic acid groups are preferably present with at least one, and more preferably at least two, carboxylic acid groups in their free acid form. The concentrations given below assume this to be the case. The enhancers in the protonated acid form are believed to not only increase the antimicrobial efficacy, but to improve compatibility when incorporated into the aliphatic polyester component.

One or more enhancers are used in the compositions of the present invention at a suitable level to produce the desired result. Enhancers are typically present in a total amount greater than 0.1 wt. %, preferably in an amount greater than 0.25 wt. %, more preferably in an amount greater than 0.5 wt. %, even more preferably in an amount greater than 1.0 wt. %, and most preferably in an amount greater than 1.5 wt. % based on the total weight of the ready-to-use degradable aliphatic polyester polymer composition. In a preferred embodiment, the enhancers are present in a total amount of no greater than 20 wt-%, or 15 wt-%, based on the total weight of the ready-to-use degradable aliphatic polyester polymer composition. Such concentrations typically apply to alpha-hydroxy acids, beta-hydroxy acids, other carboxylic acids, chelating agents, phenolics, ether glycols, and (C₅-C₁₀) monohydroxy alcohols.

The ratio of the enhancer component relative to the total concentration of the antimicrobial component is preferably within a range of 10:1 to 1:300, and more preferably 5:1 to 1:10, on a weight basis.

An alpha-hydroxy acid type of enhancer is typically a compound of the formula:

R⁶(CR¹⁷OH)ₙ₂COOH

wherein: R¹⁶ and R¹⁷ are each independently H or a (C₁-C₈) alkyl group (straight, branched, or cyclic), a (C₆-C₁₂) aryl, or a (C₆-C₁₂) aralkyl or alkaryl group (wherein the alkyl group is straight, branched, or cyclic), R¹⁶ and R¹⁷ may be optionally substituted with one or more carboxylic acid groups; and n2=1-3, preferably, n2=1-2.

Exemplary alpha-hydroxy acids include, but are not limited to, lactic acid, malic acid, citric acid, 2-hydroxybutanoic acid, mandelic acid, gluconic acid, glycolic acid, tartaric acid, alpha-hydroxyethanoic acid, ascorbic acid, alpha-hydroxyoctanoic acid, and hydroxycaprylic acid, as well as derivatives thereof (e.g., compounds substituted with hydroxyls, phenyl groups, hydroxyphenyl groups, alkyl groups, halogens, as well as combinations thereof). Preferred alpha-hydroxy acids include lactic acid, glycolic acid, malic acid, and mandelic acid. These acids may be in D, L, or DL form and may be present as free acid, lactone, or partial salts thereof. All such forms are encompassed by the term “acid.” Preferably, the acids are present in the free acid form. Other suitable alpha-hydroxy acids are described in U.S. Pat. No. 5,665,776 (Yu).

A beta-hydroxy acid enhancer is typically a compound represented by the formula:

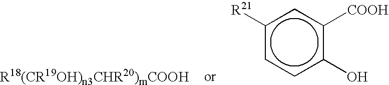

wherein: R¹⁸, R¹⁹, and R²⁰ are each independently H or a (C₁-C₈)alkyl group (saturated straight, branched, or cyclic group), (C₆-C₁₂) aryl, or (C₆-C₁₂) aralkyl or alkaryl group (wherein the alkyl group is straight, branched, or cyclic), R¹⁸ and R¹⁹ may be optionally substituted with one or more carboxylic acid groups; m=0 or 1; n3=1-3 (preferably, n3=1-2); and R²¹ is H, (C₁-C₄) alkyl or a halogen.

Exemplary beta-hydroxy acids include, but are not limited to, salicylic acid, beta-hydroxybutanoic acid, tropic acid, and trethocanic acid. In certain preferred embodiments, the beta-hydroxy acids useful in the compositions of the present invention are selected from the group consisting of salicylic acid, beta-hydroxybutanoic acid, and mixtures thereof. Other suitable beta-hydroxy acids are described in U.S. Pat. No. 5,665,776.

One or more alpha or beta-hydroxy acid enhancers may be incorporated in the degradable aliphatic polyester polymer compositions, and/or applied to the surfaces of articles comprising the degradable aliphatic polyester polymer composition, in an amount to produce the desired result. They may be present in a total amount of at least 0.25 wt-%, at least 0.5 wt-%, and at least 1 wt-%, based on the total weight of the ready-to-use composition. They may be present in a total amount of no greater than 20 wt-%, no greater than 10 wt-%, or no greater than 5 wt-%, based on the total weight of the ready-to-use degradable aliphatic polyester polymer composition.

The weight ratio of alpha or beta-hydroxy acid enhancer to total antimicrobial component is at most 50:1, at most 30:1, at most 20:1, at most 10:1, at most 5:1 or at most 1:1. The ratio of alpha-hydroxy acid enhancer to total antimicrobial component may be at least 1:120, at least 1:80, or at least 1:60. Preferably the ratio of alpha-hydroxy acid enhancer to total antimicrobial component is within a range of 1:60 to 4:1.

In systems with low concentrations of water transesterification may be the principle route of loss of the fatty acid monoester and alkoxylated derivatives of these active ingredients and loss of carboxylic acid containing enhancers may occur due to esterification. Thus, certain alpha-hydroxy acids (AHA) and beta-hydroxy acids (BHA) are particularly preferred since these are believed to be less likely to transesterify the ester antimicrobial or other ester by reaction of the hydroxyl group of the AHA or BHA. For example, salicylic acid may be particularly preferred in certain formulations since the phenolic hydroxyl group is a much more acidic alcohol and thus much less likely to react. Other particularly preferred compounds in anhydrous or low-water content formulations include lactic, mandelic, malic, citric, tartaric, and glycolic acid. Benzoic acid and substituted benzoic acids that do not include a hydroxyl group, while not hydroxyl acids, are also preferred due to a reduced tendency to form ester groups.

Carboxylic acids other than alpha- and beta-carboxylic acids are also suitable enhancers. They include alkyl, aryl, aralkyl, or alkaryl carboxylic acids typically having equal to or less than 12 carbon atoms. A preferred class of these can be represented by the following formula:

R²²(CR²³₂)ₙ₂COOH

wherein: R²² and R²³ are each independently H or a (C₁-C₄) alkyl group (which can be a straight, branched, or cyclic group), a (C₆-C₁₂) aryl group, a (C₆-C₁₂) group containing both aryl groups and alkyl groups (which can be a straight, branched, or cyclic group), R²² and R²³ may be optionally substituted with one or more carboxylic acid groups; and n2=0-3, preferably, n2=0-2. The carboxylic acid may be a (C₂-C₆) alkyl carboxylic acid, a (C₆-C₁₆) aralkyl carboxylic acid, or a (C₆-C₁₆) alkaryl carboxylic acid. Exemplary acids include, but are not limited to propionic acid, sorbic acid, benzoic acid, benzylic acid, and nonylbenzoic acid.

One or more such carboxylic acids may be used in the compositions of the present invention in amounts sufficient to produce the desired result in generally the same amounts as discussed above for the alpha or beta-hydroxy acids based on the total weight of the ready-to-use composition.

A chelating agent (i.e., chelator) is typically an organic compound capable of multiple coordination sites with a metal ion in solution. Typically these chelating agents are polyanionic compounds and coordinate best with polyvalent metal ions. Exemplary chelating agents include, but are not limited to, ethylene diamine tetraacetic acid (EDTA) and salts thereof (e.g., EDTA(Na)₂, EDTA(Na)₄, EDTA(Ca), EDTA(K)₂), sodium acid pyrophosphate, acidic sodium hexametaphosphate, adipic acid, succinic acid, polyphosphoric acid, sodium acid pyrophosphate, sodium hexametaphosphate, acidified sodium hexametaphosphate, nitrilotris(methylenephosphonic acid), diethylenetriaminepentaacetic acid, 1-hydroxyethylene, 1,1-diphosphonic acid, and diethylenetriaminepenta-(methylenephosphonic acid). Certain carboxylic acids, particularly the alpha-hydroxy acids and beta-hydroxy acids, can also function as chelators, e.g., malic acid and tartaric acid.

Also included as chelators are compounds highly specific for binding ferrous and/or ferric ion such as siderophores, and iron binding proteins. Iron binding protein include, for example, lactoferrin, and transferrin. Siderophores include, for example, enterochlin, enterobactin, vibriobactin, anguibactin, pyochelin, pyoverdin, and aerobactin.

In certain embodiments, the chelating agents useful in the compositions of the present invention include those selected from the group consisting of ethylenediaminetetraacetic acid and salts thereof, succinic acid, and mixtures thereof. Preferably, either the free acid or the mono- or di-salt form of EDTA is used.

One or more chelating agents may be used in the compositions of the present invention at a suitable level to produce the desired result. They may be used in amounts similar to the carboxylic acids described above.

The ratio of the total concentration of chelating agents (other than alpha- or beta-hydroxy acids) to the total concentration of the antimicrobial component is preferably within a range of 10:1 to 1:100, and more preferably 1:1 to 1:10, on a weight basis.

A phenolic compound enhancer is typically a compound having the following general structure:

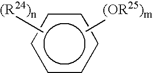

wherein: m is 0 to 3 (especially 1 to 3), n is 1 to 3 (especially 1 to 2), each R²⁴ independently is alkyl or alkenyl of up to 12 carbon atoms (especially up to 8 carbon atoms) optionally substituted with O in or on the chain (e.g., as a carbonyl group) or OH on the chain, and each R²⁵ independently is H or alkyl or alkenyl of up to 8 carbon atoms (especially up to 6 carbon atoms) optionally substituted with O in or on the chain (e.g., as a carbonyl group) or OH on the chain, but if R²⁵ is H, n preferably is 1 or 2.

Examples of phenolic enhancers include, but are not limited to, butylated hydroxy anisole, e.g., 3(2)-tert-butyl-4-methoxyphenol (BHA), 2,6-di-tert-butyl-4-methylphenol (BHT), 3,5-di-tert-butyl-4-hydroxybenzylphenol, 2,6-di-tert-4-hexylphenol, 2,6-di-tert-4-octylphenol, 2,6-di-tert-4-decylphenol, 2,6-di-tert-butyl-4-ethylphenol, 2,6-di-tert-4-butylphenol, 2,5-di-tert-butylphenol, 3,5-di-tert-butylphenol, 4,6-di-tert-butyl-resorcinol, methyl paraben (4-hydroxybenzoic acid methyl ester), ethyl paraben, propyl paraben, butyl paraben, 2-phenoxyethanol, as well as combinations thereof. One group of the phenolic compounds is the phenol species having the general structure shown above where R²⁵ is H and where R²⁴ is alkyl or alkenyl of up to 8 carbon atoms, and n is 0, 1, 2, or 3, especially where at least one R²⁴ is butyl and particularly tert-butyl, and especially the non-toxic members thereof being preferred. Some of the phenolic synergists are BHA, BHT, methyl paraben, ethyl paraben, propyl paraben, and butyl paraben as well as combinations of these.

An additional enhancer is a monohydroxy alcohol having 5-10 carbon atoms, including C₅-C₁₀ monohydroxy alcohols (e.g., octanol and decanol). In certain embodiments, alcohols useful in the compositions of the present invention are selected from the group n-pentanol, 2 pentanol, n-hexanol, 2 methylpentyl alcohol, n-octanol, 2-ethylhexyl alcohol, decanol, and mixtures thereof.

An additional enhancer is an ether glycol. Exemplary ether glycols include those of the formula:

R—O—(CH₂CHR″″O)ₙ(CH₂CHR′O)H,

wherein R═H, a (C₁-C₈) alkyl, or a (C₆-C₁₂) aralkyl or alkaryl; and each R′ is independently ═H, methyl, or ethyl; and n=0-5, preferably 1-3. Examples include 2-phenoxyethanol, dipropylene glycol, triethylene glycol, the line of products available under the trade designation DOWANOL DB (di(ethylene glycol) butyl ether), DOWANOL DPM (di(propylene glycol)monomethyl ether), and DOWANOL TPnB (tri(propylene glycol) monobutyl ether), as well as many others available from Dow Chemical Company, Midland Mich.

Oligomers that release an enhancer may be prepared by a number of methods. For example, oligomers may be prepared from alpha hydroxy acids, beta hydroxy acids, or mixtures thereof by standard esterification techniques. Typically, these oligomers have at least two hydroxy acid units, preferably at least 10 hydroxy acid units, and most preferably at least 50 hydroxy acid units. For example, a copolymer of lactic acid and glycolic acid may be prepared as shown in the Examples section.

Alternatively, oligomers of (C₂-C₆) dicarboxylic acids and diols may be prepared by standard esterification techniques. These oligomers preferably have at least 2 dicarboxylic acid units, preferably at least 10 dicarboxylic acid units.

The enhancer releasing oligomeric polyesters used typically have a weight average molecular weight of less than 10,000 daltons and preferably less than 8,000 daltons.

These oligomeric polyesters may be hydrolyzed. Hydrolysis can be accelerated by an acidic or basic environment, for example at a pH less than 5 or greater than 8. The oligomers may be degraded enzymatically by enzymes present in the composition or in the environment in which it is used, for example from mammalian tissue or from microorganisms in the environment.

Compositions of the present invention can include one or more surfactants to promote compatibility of the degradable aliphatic polyester polymer compositions and to help wet the surface and/or to aid in contacting and controlling or killing microorganisms or preventing toxin production. As used herein the term “surfactant” means an amphiphile (a molecule possessing both polar and nonpolar regions which are covalently bound) capable of reducing the surface tension of water and/or the interfacial tension between water and an immiscible liquid. The term is meant to include soaps, detergents, emulsifiers, surface active agents, and the like. The surfactant can be cationic, anionic, nonionic, or amphoteric. A variety of conventional surfactants may be used; however, it may be important in selecting a surfactant to determine that it is compatible with the finished degradable aliphatic polyester polymer compositions and does not inhibit the antimicrobial activity of the antimicrobial composition. One skilled in the art can determine compatibility of a surfactant by making the formulation and testing for antimicrobial activity as described in the Examples herein. Combinations of various surfactants can be used. Preferred surfactants are selected from the surfactants based on sulfates, sulfonates, phosphonates, phosphates, poloxamers, alkyl lactates, carboxylates, cationic surfactants, and combinations thereof and more preferably is selected from (C₈-C₂₂) alkyl sulfate salts, di(C₈-C₁₈)sulfosuccinate salts, C₈-C₂₂ alkyl sarconsinate, and combinations thereof.

One or more surfactants may be used in and/or on the degradable aliphatic polyester polymer compositions of the present invention at a suitable level to produce the desired result. In some embodiments, when used in the composition, they are present in a total amount of between about 0.1 wt. % to about 20 wt-%, based on the total weight of the degradable aliphatic polyester polymer composition.

Additionally, the compositions may further comprise organic and inorganic fillers. These materials may help to control the degradation rate of the aliphatic polyester polymer composition. For example, many calcium salts and phosphate salts may be suitable. Exemplary fillers include calcium carbonate, calcium sulfate, calcium phosphate, calcium sodium phosphates, calcium potassium phosphates, tetracalcium phosphate, .alpha.-tricalcium phosphate, beta-tricalcium phosphate, calcium phosphate apatite, octacalcium phosphate, dicalcium phosphate, calcium carbonate, calcium oxide, calcium hydroxide, calcium sulfate dihydrate, calcium sulfate hemihydrate, calcium fluoride, calcium citrate, magnesium oxide, and magnesium hydroxide. Particularly suitable filler is tribasic calcium phosphate (hydroxy apatite).

Disposable absorbent articles comprising the invention degradable aliphatic polyester polymer composition may be made by processes known in the art for making these products using sheet, webs or fibers formed from the invention degradable aliphatic polyester polymer composition. These degradable aliphatic polyester polymer compositions are used to form webs and the like that are directly formed into disposable absorbent articles without special treatments or converting processes. The degradable aliphatic polyester polymer composition webs or fibers prior to use are dry and in a stable form and remain so until in the end use environment. By dry it is meant that there is no significant added moisture and it is in equilibrium with its environment. Generally the disposable absorbent articles would be packaged in a dry environment with no added moisture and would not be exposed to moisture until opened and used by the end use consumer. When in the end use environment, upon absorption of a fluid or exposure to moisture, the antimicrobial activity of the degradable aliphatic polyester polymer composition webs or fibers is expressed and the degradable aliphatic polyester polymer composition starts or accelerates decomposition. This decomposition continues after disposal following use.

The degradable aliphatic polyester polymer compositions are particularly suitable for use in feminine tampons due to their unique combination of properties. For example, the antimicrobial compositions as described herein are particularly effective in reducing toxic shock syndrome toxin (TSST) at levels that do not necessarily kill bacteria. This allows the article to be used without killing potentially helpful bacteria but still providing protection against TSST. This is usually done at a lower loading levels of the antimicrobial composition and/or enhancer component.

The invention degradable aliphatic polyester polymer compositions have also been found to significantly reduce unpleasant odors and as such are useful in wipes or disposable absorbent garments where there is often odor generated, such as by conversion of urea to ammonia by Proteus mirabilis. The invention degradable aliphatic polyester polymer compositions also can be used to reduce microbial activity on the skin when in contact for extended periods of time. These applications are usually done at a higher loading level of the antimicrobial composition or component. The invention degradable aliphatic polyester polymer compositions can be used as an absorbent fibrous material or as additive fibers in an absorbent material or as a cover web or film adjacent an absorbent material, or as a cover web that is in contact with the skin. These uses include a topsheet for a diaper, a bed pad or a feminine pad. In these uses the invention degradable aliphatic polyester polymer compositions could be formed into a spunbond web or like nonwoven and used in a body contacting environment. In this case the loading levels should be sufficient to kill or inhibit bacterial growth over an extended period of time. The invention degradable aliphatic polyester polymer compositions when used as, in or adjacent an absorbent core can have relative high loading levels of the antimicrobial compositions to kill microbes to inhibit odor production.

Non-woven webs and sheets comprising the inventive compositions can also have good tensile strength, which is particularly important with wipe applications; and can have high surface energy to allow wettability and fluid absorbency. Additional melt additives (e.g., fluorochemical melt additive) can be added to the degradable aliphatic polyester polymer composition to decrease surface energy (increase the contact angle) and impart repellency. When repellency is desired the contact angle measured on a flat film using the half angle technique is preferably greater than 70 degrees, preferably greater than 80 degrees and most preferably greater than 90 degrees.

The rate of release of antimicrobial components from the aliphatic polyester may be affected by incorporation of plasticizers, surfactants, emulsifiers, enhancers, humectants, wetting agents as well as other components. Suitable humectants and/or wetting agents may include polyhydric alcohols such as polypropylene glycol and polyethylene glycol.

The level of antimicrobial activity in a given use environment is related to the finished composition, including the weight percents of the antimicrobial component and the enhancer, as well as the presence and weight percent of additional components such as surfactants and wetting agents. The level of antimicrobial activity is also related to the amount of the invention degradable thermoplastic aliphatic polyester material that is present in the disposable absorbent articles as well as where and how the material is incorporated into the disposable article. An additional aspect potentially impacting the level of antimicrobial activity is the total surface area of the degradable thermoplastic aliphatic polyester within the disposable absorbent article. Thus one way to increase the antimicrobial activity as a given weight of degradable thermoplastic aliphatic polyester material within a disposable absorbent article is to use nonwovens or fibers with a smaller fiber diameters, and thus more surface area per unit weight.

In a preferred embodiment the articles of the present invention are kept dry until use. This protects the aliphatic polyester from potential degradation as well as any antimicrobial ester that may be present from hydrolytic degradation. The amount of moisture present is preferably low. Typically, the amount of water in the packaged article prior to use is less than 10% by weight, preferably less than 8% by weight and usually less than 5% by weight. Packaging may be used that protects the article from absorbing moisture in humid environments. For example, the articles may be packaged with a protective film of polyolefin, polyester (e.g. polyethylene terephalate, polyethylene naphthylate etc.), fluoropolymers (e.g. Aclar available from Allied Signal Morristown, Pa.), PVDC, PVC, ceramic barrier coated films, as well as laminates and blends thereof.

In one process for making the inventive antimicrobial composition, the aliphatic polyester in a melt form is mixed in a sufficient amount relative to the antimicrobial component to yield an aliphatic polyester polymer composition having measurable antimicrobial activity. An enhancer and optionally a surfactant can be added to the melt of the aliphatic polyester polymer composition and/or coated on the surface of an article comprising the degradable aliphatic polyester polymer composition to enhance the antimicrobial component.

A variety of equipment and techniques are known in the art for melt processing aliphatic polyester polymeric compositions. Such equipment and techniques are disclosed, for example, in U.S. Pat. No. 3,565,985 (Schrenk et al.), U.S. Pat. No. 5,427,842 (Bland et. al.), U.S. Pat. Nos. 5,589,122 and 5,599,602 (Leonard), and U.S. Pat. No. 5,660,922 (Henidge et al.). Examples of melt processing equipment include, but are not limited to, extruders (single and twin screw), Banbury mixers, and Brabender extruders for melt processing the degradable aliphatic polyester polymer composition. To maximize the antimicrobial activity of any given degradable thermoplastic aliphatic polyester composition at a given weight of inclusion in a disposable absorbent article it may be desirable to use fibers with very small fiber diameters, such as micro or nanofibers. Methods of producing nanofibers with thermoplastic materials are known, for example as taught in U.S. Pat. Nos. 4,536,361, 6,382,526, and 6,695,992. It is also known to make polylactic acid based micro and nanofibers, and nonwoven webs of such fibers, using various methods, e.g. as taught in U.S. Patent Application 2006/0084340 A1. Thus for some disposable absorbent articles of the present invention it may be preferred to make the article with nonwovens and/or fibers of degradable thermoplastic aliphatic polyester composition wherein the fiber diameter is about 1 micron or preferably less.

The ingredients of the degradable thermoplastic aliphatic polyester composition may be mixed in and conveyed through an extruder to yield a material having measurable antimicrobial activity, preferably without polymer degradation or side reactions in the melt. The processing temperature is sufficient to mix the biodegradable aliphatic polyester and antimicrobial component, and allow extruding the composition as a film, nonwoven or fiber. Potential degradation reactions include transesterification, hydrolysis, chain scission and radical chain decomposition, and process conditions should minimize such reactions.

The invention will be further clarified by the following examples which are exemplary and not intended to limit the scope of the invention.

### EXAMPLES

### Examples 1 and 2

Samples were prepared using a batch Brabender mixing apparatus in which pelletized polylactic acid (PLA polymer obtained from NatureWorks LLC as Polymer 4032 D and 4060 D) was added to the Brabender mixer and blended at 180° C. until the mixing torque stabilized. The other ingredients were then added to the mixer, and the total composition was blended until it appeared homogeneous. The mixture was then pressed into sheets using a hydraulic press the platens of which were at the 177° C. Samples of the sheets were tested for microbial activity using Japanese Industrial Standard test number Z 2801: 2000 using a Gram-positive bacteria (Staphylococcus aureus ATCC #6538) and a Gram-negative bacteria (Pseudomonas aeruginosa ATCC #9027). The same test was performed on a control sheet of polylactic acid without the added ingredients. The data from this testing is presented in Table 1 below.

### Antimicrobial Testing of Film Samples:

The following test protocol, adapted from JIS Z2801 (Japanese Industrial Standard—Test for Antimicrobial Activity), was used to assess antimicrobial properties of extruded or pressed films. Approximately 4 cm×4 cm squares of test material were wiped with isopropanol or 70% ethanol and placed into sterile Petri dishes. Duplicate test samples were each inoculated with 0.4 mL of challenge organisms (Staphlyococcus aureus ATCC #6538 or Pseudomonas aeruginosa ATCC #9027 diluted 1:5000 from overnight cultures into 0.2% TSB). 2 cm×2 cm squares of polyester film were then placed onto the inoculum. Samples were then incubated 18-24 h at 37° C. in 80% relative humidity or higher. After incubation, test samples were removed from the Petri dishes and each transferred into 10 mL sterile Difco Dey Engley Neutralizing Broth (NB). The tubes containing the NB and test material were placed into an ultrasonic bath for 60 s then mixed for 60 s to release the bacteria from the materials into the NB. Viable bacteria were then enumerated by diluting the NB into phosphate-buffered saline (PBS), plating onto TSB agar, incubating plates at 37° C. for 24-48 h, and counting colony forming units (CFUs). Sensitivity limit for this test method was deemed to be 100 CFU/sample.

TABLE 1

| | | | | | Microbe | |
|---|---|---|---|---|---|---|
| | | | | | Count (cfu/ml) | |
| | | PML | | | P. | S. |
| Sample | PLA (g) | (ml) | BA (g) | DOSS (g) | aeruginosa | aureus |
| PLA - | 55 | 0 | 0 | 0 | >10⁷ | >10⁵ |
| Control 1 | (4032D) | | | | | |
| Example 1 | 55 | 5 | 1 | 1 | <100 | <100 |
| | (4060D) | | | | | |
| Example 2 | 55 | 9 | 1 | 0 | <100 | <100 |
| | (4032D) | | | | | |

PML means propyleneglycol monolaurate antimicrobial component, obtained from Abitec Corp., as Capmul PG12.
BA means benzoic acid enhancer
DOSS means dioctylsulfosuccinate sodium salt surfactant.
PLA 4032D is semicrystalline polylactic acid from Natureworks LLC.
PLA 4060D is amorphous polylactic acid from Natureworks LLC.

The above data show the broad-spectrum efficacy of the degradable aliphatic polyester polymer composition in sheet form in killing both a Gram-positive and a Gram-negative bacteria.

### Preparation of Oligomeric Lactic Acid Enhancer and Master Batches:

An oligomeric enhancer was used in Examples 3-14 and was prepared using the following procedure. A glass reactor (ambient pressure) was filled with equal parts of an 85% lactic acid aqueous solution (City Chemicals) and a 70% glycolic acid aqueous solution (Sigma-Aldrich). The water boiled was boiled away leaving the acid monomers. Reactor temperature was then increased to 163° C. initiating a condensation polymerization of the lactic and glycolic acids. Reaction was allowed to proceed for 24 hours resulting in a random copolymer or oligomer of the two acids with a molecular weight of 1,000-8,000 M_{w} for one batch and 700-1,000 M_{w} for another batch.

Pre-compounded pellets, used in Examples 3-14 were prepared with a Werner Pfleiderer ZSK-25 twin screw extruder. The extruder had ten zones, each having a barrel section with a channel for circulating heat transfer fluid, and all but the first (feed) section having heating elements. The screw configurations were helical conveying screw sections, except that kneading sections were used in the second half of zone 2, first half of zone 3, all of zone 5, first half of zone 6, all of zone 8 and the first half of zone 9. Extruder vent plugs at zones 5 and 9 were plugged. Pellets of polylactic acid PLA 625 ID (Natureworks LLC) were added to the first zone of the extruder at a rate of 3.6 kg/hr. Antimicrobial fatty acid monoester was pumped into the fourth zone of the extruder using a Dynatec S-05 model grid-melter at a rate of 0.5 kg/hr. The grid-melter used a gear pump to meter liquid monoester through transfer tubing into the extruder. The pump and tubing were operated at room temperature when using propylene glycol monolaurate and at 70° C. when using glycerol monolaurate. The oligomeric enhancer described above was heated to 120° C. in a heated tank and gravity fed to a metering pump which delivered it to zone 7 of the extruder at a rate of 0.5 kg/hr. A metering pump was employed at the discharge of the extruder to feed a strand die having a 6.35 mm diameter opening. The extruded strand was cooled in an 2.4 meter long water trough (with continuously fed tap water) and then, at the outlet of the water bath, pelletized using a Conair pelletizer into approximately 6.35 mm length pellets. The extruder screw speed was maintained at 100 RPM and the following barrel temperature profile was used: zone 1-160° C.; zone 2-200° C.; zone 3-177° C.; zones 4 through 9-160° C. The metering pump was electrically heated and adjustable to a temperature set point, set at 177° C., and pump speed was adjusted manually to maintain a pressure of approximately 70-140 N/cm² (100-200 lbs/in²) to the inlet of the melt pump.

Three masterbatches were prepared having the compositions listed below. The pellets were dried in a forced air resin drier with frequent stirring to prevent agglomeration of the pellets.

Masterbatch #1: 80% PLA 6251D, 10% glycerol monolaurate (GML) & 10% oligomeric enhancer (OLGA).
Masterbatch #2: 80% PLA 6251 D, 10% propyleneglycol monolaurate (PML) & 10% oligomeric enhancer (OLGA).
Masterbatch #3: 90% PLA 6251 D & 10% glycerol monolaurate (GML).

### Examples 3-5

Blown microfiber nonwoven webs were produced from the masterbatches described above using conventional melt blowing equipment. A 31 mm (screw diameter) conical twin screw extruder (C.W. Brabender Instruments) was used to feed a positive displacement gear pump which was used to meter and pressurize the aliphatic polyester polymer melt. A 25 cm wide drilled orifice melt-blowing die with 8 orifices per cm of width was used. Each orifice was 0.38 mm in diameter. Extruder temperature was 185° C., die temperature was 180° C., air heater temperature was 200° C., and air manifold pressure was 103 kPa. Total polymer flow rate through the die was approximately 3.6 kg/hr. A control sample, Control 2 was prepared containing no enhancer or antimicrobial component. A control sample, Control 3, was also prepared containing no enhancer but having an antimicrobial component. For samples having lower than 10% enhancer or antimicrobial additive, additional virgin PLA resin was added to the masterbatch. Characteristics of the nonwoven webs are shown in Table 2 below.

TABLE 2

| | | | Basis | Web | Effective Fiber |
|---|---|---|---|---|---|
| | | % wt | Weight | thickness | Diameter* |
| Sample | % wt GML | OLGA | (g/m²) | (mm) | (μm) |
| Control 2 | 0 | 0 | 92 | 1.7 | 22.8 |
| Control 3 | 10 | 0 | 95 | 1.3 | 20.7 |
| Example 3 | 10 | 10 | 107 | 0.7 | 10.7 |
| Example 4 | 5 | 5 | 94 | 1.1 | 14.9 |
| Example 5 | 2.5 | 2.5 | 95 | 1.4 | 20.1 |

*Effective Fiber Diameter (in micrometers) was calculated as described by Davies, C. N., “The Separation of Airborne Dust and Particles”, Institution of Mechanical Engineers, London Proceedings 1B, 1952.

### Examples 6-8

Blown microfiber nonwoven webs were produced as in Examples 3-5 except propyleneglycol monolaurate (PML) was used as the antimicrobial component. Characteristics of the nonwoven webs are shown in Table 3 below.

TABLE 3

| | | | Basis | Web | Effective Fiber |
|---|---|---|---|---|---|
| | | % wt | Weight | thickness | Diameter* |
| Sample | % wt PML | OLGA | (g/m²) | (mm) | (μm) |
| Example 6 | 10 | 10 | 103 | 0.8 | 12.5 |
| Example 7 | 5 | 5 | 95 | 1.1 | 15.4 |
| Example 8 | 2.5 | 2.5 | 94 | 1.1 | 14.9 |

Examples 3-5 and Control 2 and Control 3 were tested for tensile strength and stiffness properties. Peak force tensile strength was measured using an INSTRON Model 5544 universal tensile testing machine using a crosshead speed of 25.4 cm/min with a gauge length of 5.1 cm. The specimen dimensions were 10.2 cm in length. Machine (MD) and cross (CD) directions of the nonwoven webs were tested. The percent elongation of the specimen at peak force was recorded. Ten replicates were tested and averaged for each sample web. Results are shown below in Table 4.

Stiffness properties of the webs were measured using a Gurley bending resistance tester model 4151E (Gurley Precision Instruments). 3.8 cm long by 2.5 cm wide specimens were cut from the webs, the long direction being in the machine direction of the web. Each specimen was tested by deflecting the specimen in both the MD and CD and calculating the average of both directions of the pendulum deflections. The tester was used to convert the pendulum deflection measurements and machine settings to Gurley stiffness readings in milligrams. Ten replicates were tested and averaged for each sample web. Results are shown below in Table 4.

TABLE 4

| | Peak | | | | |
|---|---|---|---|---|---|
| | Force | | Peak Force | | |
| | MD (g/cm | Elongation | CD (g/cm | Elongation | Stiffness |
| Sample | width) | MD (%) | width) | CD (%) | (mg) |
| Control 2 | 66 | 15.8 | 93 | 102.3 | 126 |
| Control 3 | 120 | 11.4 | 129 | 90.1 | 100 |
| Example 3 | 813 | 6.8 | 620 | 7.8 | 507 |
| Example 4 | 377 | 2.8 | 375 | 75.8 | 346 |
| Example 5 | 193 | 15.3 | 188 | 81.5 | 113 |

TABLE 5
(AATCC 100-2004 Antibacterial testing using
Staphlyococcus aureus)

| Sample | CFU/ml | CFU/sample |
|---|---|---|
| t = 0 | 80000 | 1600000 |
| Control 2 | 42000 | 840000 |
| Control 3 | <200 | <200 |
| Example 3 | <200 | <200 |
| Example 4 | <200 | <200 |
| Example 5 | 230 | 4600 |

TABLE 6
(AATCC 100-2004 Antibacterial testing using
Pseudomonas aeruginosa)

| Sample | CFU/ml | CFU/sample |
|---|---|---|
| t = 0 | 34000 | 680000 |
| Control 2 | 2600000 | 52000000 |
| Control 3 | 2200 | 44000 |
| Example 3 | <200 | <200 |
| Example 4 | <200 | <200 |
| Example 5 | 330000 | 6600000 |

TABLE 7
(Log Reduction vs. t = 0), summary of results presented in
Table 5 and 6

| Sample | Staphlyococcus aureus | Pseudomonas aeruginosa |
|---|---|---|
| Control 2 | 0.5 | −1.6 |
| Control 3 | 3.9 | 1.2 |
| Example 3 | 3.9 | 3.5 |
| Example 4 | 3.9 | 3.5 |
| Example 5 | 2.5 | −1.0 |

Table 7 was calculated by taking the log of the quotient of the time-zero CFU/sample count by the final CFU/sample count.

TABLE 8
(AATCC 100-2004 Antibacterial testing using
Staphlyococcus aureus)

| Sample | CFU/ml | CFU/sample |
|---|---|---|
| t = 0 | 130000 | 2600000 |
| Control 2 | 42000 | 840000 |
| Example 6 | <200 | <200 |
| Example 7 | <200 | <200 |
| Example 8 | 15 | 300 |

TABLE 9
(AATCC 100-2004 Antibacterial testing using
Pseudomonas aeruginosa)

| Sample | CFU/ml | CFU/sample |
|---|---|---|
| t = 0 | 70000 | 1400000 |
| Control 2 | 2600000 | 52000000 |
| Example 6 | <200 | <200 |
| Example 7 | <200 | <200 |
| Example 8 | 25 | 500 |

TABLE 10
(Log Reduction vs t = 0), summary of results presented in
Tables 8 and 9

| Sample | Staphlyococcus aureus | Pseudomonas aeruginosa |
|---|---|---|
| Control 2 | 0.5 | −1.6 |
| Example 6 | 4.1 | 3.8 |
| Example 7 | 4.1 | 3.8 |
| Example 8 | 3.9 | 3.4 |

Table 10 was calculated by taking the log of the quotient of the time-zero CFU/sample count by the final CFU/sample count.

The results presented in Tables 5-10 demonstrate the broad-spectrum efficacy of example compositions against both a Gram-positive and a Gram-negative bacteria.

### Examples 9-13

Spunbond nonwoven examples were prepared using masterbatch prepared as described above blended with neat PLA to prepare examples 9-13. The compositions of these masterbatches were: 20% PML in PLA, 30% OLGA In PLA, and 10% PEG 400 in PLA. The PLA used to make these masterbatches was PLA 6202D and the percentages reported are weight percentages of the component in the masterbatch composition. The OLGA used was prepared as described above and had a molecular weight (M_{w}) of about 1000.

These examples were prepared with PLA 6202D resin obtained from NatureWorks, LLC. Propylene glycol monolaurate trade name Capmul PG-12 was obtained from ABITEC Corporation. Master-batches of the PLA and the additives were compounded using the procedure described above for the masterbatches used for Examples 3-8. All the materials were dried prior to use. The spunbond nonwovens were obtained using a 2.0 inch single screw extruder to feed a die. The die had a total of 512 orifice holes with a aliphatic polyester polymer melt throughput of 0.50 g/hole/min (33.83 lb/hr). The die had a transverse length of 7.875 inches (200 mm). The hole diameter was 0.040 inch (0.889 mm) and L/D ratio of 6. The melt extrusion temperature of the neat PLA was set at 215° C., while the melt extrusion temperature of PLA with the additives was dependent on the amount of additives: Example 9 (185° C.), Examples 10-12 (175° C.), and Example 13 (162° C.).

The compositions of the spunbond nonwoven examples prepared are described in Table 11. In addition to the examples including propylene glycolmonolaurate as the antimicrobial component of the antimicrobial composition and OLGA as the enhancer component one example also included polyethylene glycol as a wetting agent, Also a control example spunbond nonwoven, Control 4, was prepared comprising only PLA, Some physical properties of the examples of Table 11 are described in Table 12.

TABLE 11
Spunbond nonwoven samples

| | PLA | | | |
|---|---|---|---|---|
| | Weight | PML | OLGA | Wetting Agent/ |
| Sample | Percent | Weight Percent | Weight Percent | Weight Percent |
| Control 4 | 100% | 0% | 0% | 0% |
| Example 9 | 90% | 5% | 5% | 0% |
| Example 10 | 85% | 5% | 10% | 0% |
| Example 11 | 83% | 5% | 10% | 2% |
| Example 12 | 80% | 5% | 15% | 0% |
| Example 13 | 75% | 5% | 20% | 0% |

The wetting agent used in Example 11 was polyethylene glycol 400

TABLE 12
Physical characteristic of spunbond nonwoven samples

| | | Fiber |
|---|---|---|
| | Basis Weight | Diameter* |
| Sample | (g/m2) | (μm) |
| Control 4 | 50 | 15.0 |
| Example 9 | 50 | 13.3 |
| Example 10 | 50 | 14.4 |
| Example 11 | 50 | 11.2 |
| Example 12 | 50 | 10.5 |
| Example 13 | 50 | 12.4 |

*measurement of 10 fibers at 200 x

### Antimicrobial and Odor Reduction Testing for Spunbond Nonwoven Examples

### Time-Kill Method:

The following test protocol, adapted from AATCC 100-2004 (Assessment of Antibacterial Finishes on Textile Materials), was used to assess antimicrobial properties of the nonwoven webs. Approximately 4×4 cm squares of test material were placed into sterile Petri dishes. Duplicate test samples were each inoculated with 1 ml of challenge organisms (Staphlyococcus aureus ATCC #6538 or Pseudomonas aeruginosa ATCC #9027 diluted 1:5000 from overnight cultures into 0.2% [v/v] tryptic soy broth (TSB) or Proteus mirabilis ATCC #14153 diluted 1:5000 into artificial urine [Sarangapani et al., J. Biomedical Mat. Research 29:1185]). Samples were then incubated 18-24 h at 37° C. in 80% relative humidity or higher. After incubation, test samples were removed from the Petri dishes and each transferred into 20 mL sterile Difco Dey Engley Neutralizing Broth (NB). The tubes containing the NB and test material were placed into an ultrasonic bath for 60 s then mixed for 60 s to release the bacteria from the materials into the NB. Viable bacteria were then enumerated by diluting the NB into phosphate-buffered saline (PBS), plating onto TSB agar, incubating plates at 37° C. for 24-48 h, and counting colony forming units (CFUs). Sensitivity limit for this test method was 200 CFU/sample.

### Odor Control Testing Method:

Overnight culture of Proteus mirabilus ATCC #14153 was diluted 1:50,000 into artificial urine (prepared according to Sarangapani et al., J. Biomedical Mat. Research 29:1185) with 5% [v/v] TSB to achieve a cell concentration of approximately 10⁶ per mL. 5 mL of this inoculum was pipetted onto approximately 1 g non-woven materials in 100 mL Pyrex jars. The bottles were sealed and incubated for 24 h at 37° C. Four people were asked to briefly open the jars under their noses and smell for ammonia odor. In some experiments, samples were inoculated with a more dilute suspension of bacteria, approximately 10³ per mL. In some experiments, bovine serum albumin (BSA) was added to 1% in the artificial urine to determine material efficacy in the presence of additional protein. In some experiments, remaining viable bacteria in the samples were measured by adding 50 mL NB to the samples which were then ultrasonically mixed in a water bath for 10 min. Dilutions of these samples were plated out on TSB agar, incubated overnight at 37° C. and CFUs counted.

### TSST-1 Inhibition: Nonwoven Extracts

4.5 g of indicated nonwoven examples were incubated approximately 24 h in 100 mL PBS at 37° C. w/shaking to obtain an extract. Brain-heart infusion (BHI, Difco) was added to the extracts to achieve final concentration of 1×BHI. These extracts with BHI were sterile filtered using a 0.2 μm pore size membrane. Five mL of the extracts with BHI were inoculated with an overnight culture of TSST-producing S. aureus strain FRI1169 diluted 1:500. After incubation with shaking at 37° C. for 24 h, cultures were centrifuged at 3200×g for 10 min to remove cells and the supernatant tested for TSST according to the Toxin Technology (Sarasota, Fla.) TSST EIA kit directions.

### TSST Inhibition: Tampon Sac Method

The following test protocol was adapted from the tampon sac method described by Reiser et al. (J. Clin. Microbiol. 25:1450). Dry test materials were added to rinsed dialysis membrane (Spectra/Por, 10,000 molecular weight cut-off, 32 mm width) and immersed in approximately 50° C. molten 1% brain-heart infusion (BHI) agar. The membranes had been inoculated with 100 μl of an overnight culture of TSST-producing S. aureus strain FRI1169 diluted to approximately 10⁶ cells per mL. Weights of test material equivalent to commercially available tampon weight were used. After 24 h incubation, samples were removed, their weight gain measured, and were placed into a zip-loc bag and sterile phosphate-buffered saline added to bring total weight gain up to 4× that of the dry weight. Fluid was extracted by kneading the test material in the zip-loc bag for approximately one minute. The resulting extract was diluted and plated for viable count and TSST was quantified according to the Toxin Technology TSST EIA kit directions.

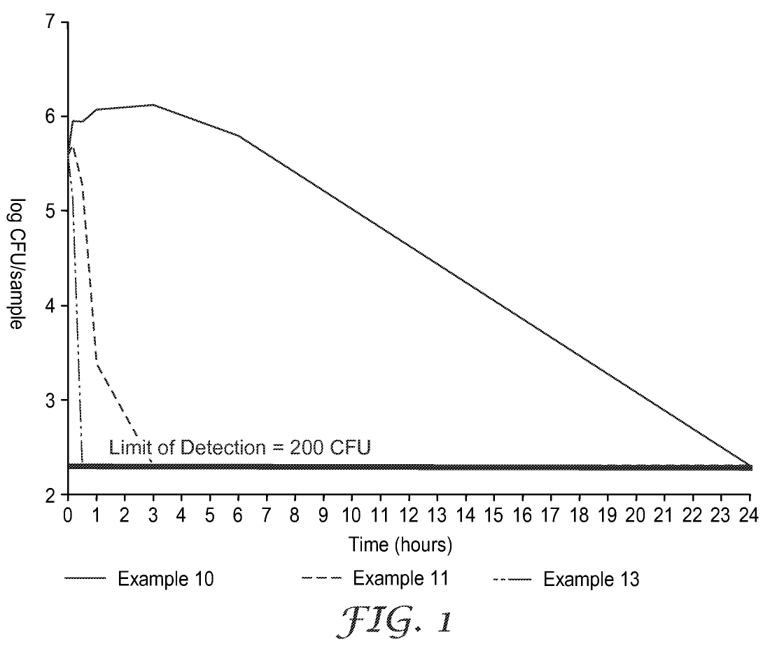

FIG. 1 shows antimicrobial activity of Examples 10, 11 and 13 against Staphlyococcus aureus using method AATCC 100. The time-kill curves exemplify the tunable nature of the antimicrobial polymer system. The ratio of the antimicrobial composition components can be adjusted to slowly reduce viable microorganisms over time or to quickly reduce the number of viable organisms to undetectable levels. The values represent averages from duplicate samples.

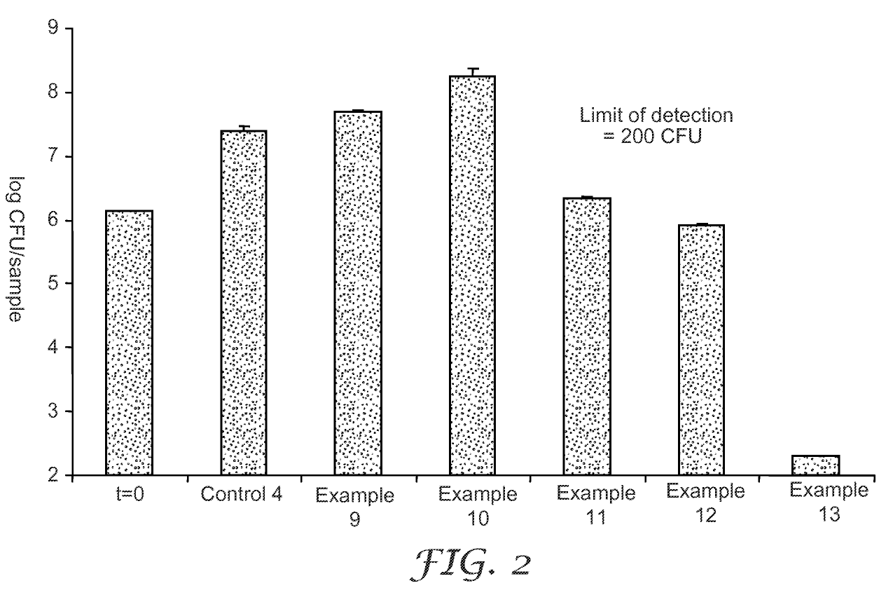

FIG. 2 shows the viable P. mirabilis recovered from Examples 9-13 after 24 hours when challenged with high numbers of the organism in the presence of artificial urine using modified method AATCC 100. The data illustrate that the composition of the antimicrobial polymer can be tuned to either inhibit growth without significantly reducing the number of viable microorganisms or to kill microorganisms even when challenged with relatively high numbers of microorganisms (approximately 10⁶ CFU/sample). Whereas Control 4 and Examples 9 and 10 allowed growth of P. mirabilis as compared to the initial inoculum (t=0), Examples 11 and 12 inhibited growth, and Example 13 reduced viable P. mirabilis to undetectable levels. The values represent averages from duplicate samples.

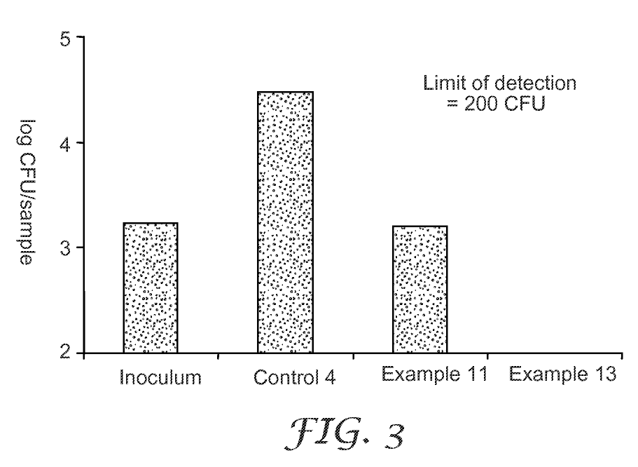

FIG. 3 shows the viable P. mirabilis recovered from Examples 11 and 13 after 24 hours when challenged with low numbers of the organism in the presence of artificial urine using modified method AATCC 100. The data illustrate that the composition of the antimicrobial polymer can also be tuned to either inhibit growth or to kill microorganisms when challenged with a low inoculum of organisms (approximately 10³ CFU/sample). Whereas Control 4 allowed growth of P. mirabilis as compared to the initial inoculum, Example 11 inhibited growth and Example 13 reduced viable P. mirabilis to undetectable levels.

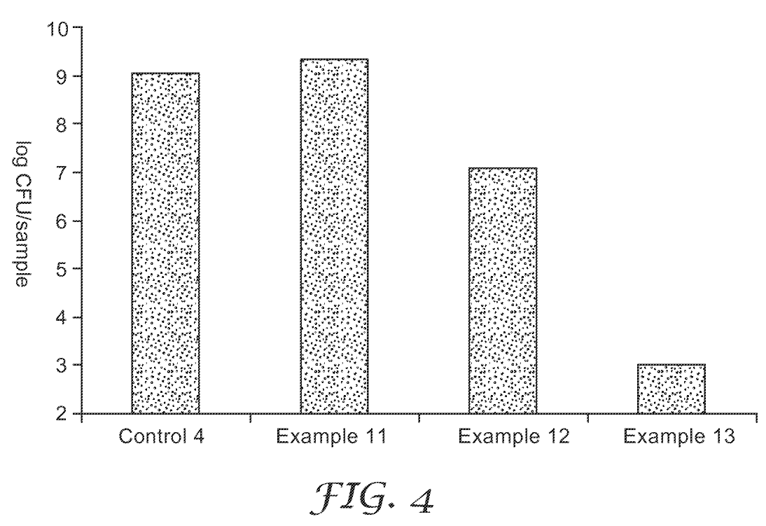

FIG. 4 shows the viable P. mirabilis recovered after odor testing of Examples 11-13 in the presence of artificial urine are reduced when exposed to certain ratios of the antimicrobial composition components. The reduced number of viable bacteria recovered from Examples 12 and 13 correlates with the lack of odor in these samples (Table 13).

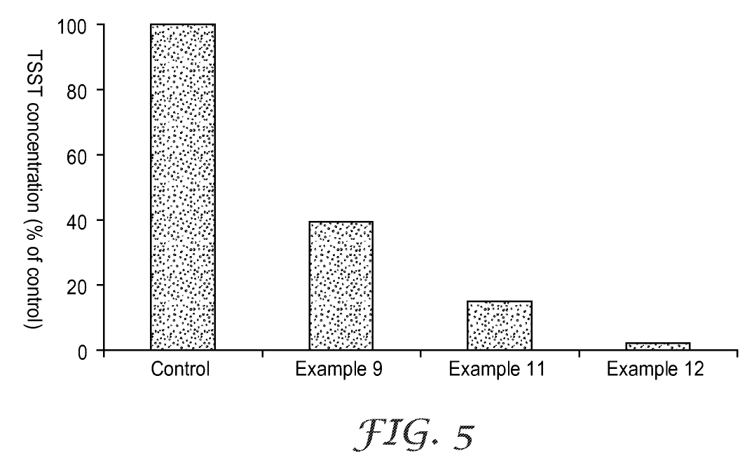

FIG. 5 shows TSST production by S. aureus incubated in the presence of extracts from material examples adjusted for toxin production per optical density unit and expressed as a percentage of TSST produced in a control culture with no added extract. The data demonstrate that TSST production is reduced when S. aureus cultures are grown in the presence of extracts from antimicrobial polymer examples. The ratio of the antimicrobial composition components can be adjusted such that toxin production is nearly eliminated as compared to a control S. aureus culture containing no extract from the antimicrobial polymers. There was little effect of the extracts on growth of the S. aureus cultures, with less than two-fold difference in optical density among all cultures shown (data not shown).

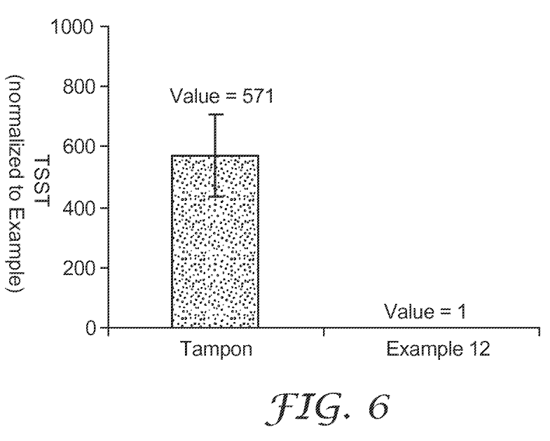

FIG. 6 shows reduced TSST production by S. aureus in Example 12 compared to a standard tampon when tested using the tampon sac method. Values are normalized to TSST produced in Example 12 and are averages of three replicates.

TABLE 13
Odor Testing Results

| | High Inoculum | Low Inoculum | High Inoculum + |
|---|---|---|---|
| Sample | (10⁶) | (10³) | BSA |
| Control 4 | + | + | + |
| Example 9 | | − | |
| Example 10 | | − | |
| Example 11 | + | − | |
| Example 12 | − | | − |
| Example 13 | − | | |

The results in Table 13 demonstrate the efficacy of the material examples in controlling odor using the described method (+ indicating strong odor and − indicating little or now odor). This efficacy is maintained even in the presence of higher protein concentrations (such as BSA) that may neutralize other antimicrobial chemistries. A higher ratio of the antimicrobial composition to the overall polymer composition may be required to control high numbers of organisms, while lower ratios may be sufficient to control lower numbers of organisms.

### Examples 14

Antimicrobial extruded films were produced using the following procedure. The co-rotating twin screw extruder, used to compound masterbatch pellets described above, was used to melt, blend and feed the aliphatic polyester polymer and additives. The screw sections were set up with kneading blocks at zones 2, 4 and 6. The extruder had 9 temperature controllable barrel zones, with an input port for dry pellets at zone 1 and liquid injection ports at zones 3 and 5. A weight loss gravimetric feeder (K-tron) was used to feed dry pellets at zone 1. 4032D semicrystalline polylactic acid (PLA) (Natureworks LLC) pellets were first dried overnight at 60° C. in a resin dryer. A grid-melter, (Dynatec) was used to melt and feed propylene glycol monolaurate (PML), (Capmul PG-12, Abitec), into zone 3 of the extruder. A metering pump (Zenith pump), was used to feed enhancer (OLGA) into zone 5 of the extruder. The enhancer was gravity fed from a heated pot directly above the pump. The melt from the extruder was fed to a metering pump, and then into a 15.24 cm wide coat-hanger die. The extrudate was extruded horizontally onto a 15.24 cm diameter temperature controlled roll. The resulting web was pulled around the roll at a 270° wrap angle. The web was then wrapped around a second 15.2 cm diameter temperature controlled roll at a 180° wrap. The web was then pulled with a nip and wrapped onto a core. Film caliper was measured with a micrometer to the nearest 2.5 microns. Film caliper was maintained to +/−15 microns using die adjustment bolts. The compositions of the films are shown below in Table 14.

TABLE 14

| Sample | PLA % | PML % | OLGA % |
|---|---|---|---|
| Control 5 | 100 | 0 | 0 |
| Example 14 | 80 | 10 | 10 |
| Control 6 | 90 | 10 | 0 |
| Control 7 | 90 | 0 | 10 |

### Example 15

Extruded films were prepared as in Examples 14 except polycaprolactone (PCL, type FB 100, Solvay Chemicals) was used as the base aliphatic polyester polymer. The compositions of the films are shown below in Table 15.

TABLE 15

| Sample | PCL % | PML % | OLGA % |
|---|---|---|---|
| Control 8 | 100 | 0 | 0 |
| Example 15 | 90 | 5 | 5 |

Antimicrobial properties of the extruded films are shown in Tables 16, 17 and 18 below.

TABLE 16
(Antibacterial testing using Staphlyococcus aureus)

| Sample | CFU/ml | CFU/sample |
|---|---|---|
| t = 0 | 39000 | 390000 |
| Control 5 | 4950 | 49500 |
| Example 14 | <100 | <100 |
| Control 6 | 1150 | 11500 |
| Control 7 | 4500 | 45000 |
| Control 8 | 490000 | 4900000 |
| Example 15 | 0 | 0 |

Values of 0 in Tables 16-17 indicate results below the detection limit of the test: approximately 100 CFU/sample.

These results show that the addition of the PML without enhancer (Control 6) reduces the Gram-positive bacteria counts over the control (Control 5). The addition of OLGA without antimicrobial component had little antimicrobial effect (Control 7). However, the addition of both PML and OLGA (Examples 14 and 15, produced a composition with exceptional antimicrobial activity, reducing the viable bacteria to levels below detection.

TABLE 17
(Antibacterial testing using Pseudomonas aeruginosa)

| Sample | CFU/ml | CFU/sample |
|---|---|---|
| t = 0 | 72000 | 720000 |
| Control 5 | 1650000 | 16500000 |
| Example 14 | <100 | <100 |
| Control 6 | 8000000 | 80000000 |
| Control 7 | 1262500 | 12625000 |
| Control 8 | 3700000 | 37000000 |
| Example 15 | <100 | <100 |

These results show that the addition of the PML without enhancer (Control 6) did not reduce Gram-negative bacteria counts over the control (Control 5). The addition, of OLGA without antimicrobial component had little antimicrobial effect (Control 7). However, the addition of both PML and OLGA (Examples 14 and 15) produced a composition with exceptional antimicrobial activity, reducing the viable bacteria to levels below detection.

TABLE 18
(Log reduction versus t = 0), summary of results from
Tables 16 and 17

| Sample | Staphlyococcus aureus | Pseudomonas aeruginosa |
|---|---|---|
| Control 5 | −0.1 | −1.4 |
| Example 14 | 3.6 | 3.9 |
| Control 6 | 1.5 | −2.0 |
| Control 7 | 0.9 | −1.2 |
| Control 8 | −1.1 | −1.7 |
| Example 15 | 3.6 | 3.9 |

Table 18 was calculated by taking the log-base-10 of the quotient of the time-zero CFU/sample count by the final CFU/sample count.

While certain representative embodiments and details have been discussed above for purposes of illustrating the invention, various modifications may be made in this invention without departing from its true scope, which is indicated by the following claims.

What is claimed is:

1. A dry delivered disposable absorbent article formed with a degradable thermoplastic aliphatic polyester composition, including an antimicrobial composition, comprising a fibrous absorbent material and one or more components formed from a degradable thermoplastic aliphatic polyester composition in the form of an extruded web or fibers of:
a) a thermoplastic aliphatic polyester;
b) an antimicrobial component incorporated therein, selected from the group consisting of: (C₇-C₁₄) saturated fatty acid esters of a polyhydric alcohol, (C₇-C₂₂) unsaturated fatty acid esters of a polyhydric alcohol, (C₇-C₁₄) saturated fatty ethers of a polyhydric alcohol, (C₈-C₂₂) unsaturated fatty ethers of a polyhydric alcohol, (C₂-C₈) hydroxy acid esters of (C₇-C₂₂) alcohols, alkoxylated derivatives thereof, and combinations thereof, wherein the alkoxylated derivatives have less than 5 moles of alkoxide group per mole of polyhydric alcohol; with the proviso that for polyhydric alcohols other than sucrose, the esters comprise monoesters and the ethers comprise monoethers, and for sucrose the esters comprise monoesters, diesters, or combinations thereof, and the ethers comprise monoethers, diethers, or mixtures thereof, wherein the antimicrobial component is present in an amount greater than 1 percent by weight of the aliphatic polyester; and
c) an enhancer selected from the group consisting of alpha-hydroxy acids, beta-hydroxy acids, chelating agents, (C₂-C₆) saturated or unsaturated alkyl carboxylic acids, (C₆-C₁₆) aryl carboxylic acids, (C₆-C₁₆) aralkyl carboxylic acids, (C₆-C₁₂) alkaryl carboxylic acids, phenolic compounds, (C₁-C₁₀) alkyl alcohols, ether glycols, oligomers that degrade to release one of the aforesaid enhancers, and mixtures thereof in an amount greater than 0.1 percent by weight of the aliphatic polyester, except for phenolic compounds which are in an amount greater than 0.5 weight percent wherein the antimicrobial composition is formed by the antimicrobial component and enhancer.

2. The disposable absorbent article of claim 1 wherein provided that, if the antimicrobial component is selected from (C₈-C₁₂) saturated fatty acid esters of a polyhydric alcohol, (C₈-C₁₈) unsaturated fatty acid esters of a polyhydric alcohol, or alkoxylated derivatives thereof, the purity of the antimicrobial component exceeds 85 percent by weight monoester.

3. The disposable absorbent article of claim 1 the degradable thermoplastic aliphatic polyester composition further comprising a surfactant distinct from the antimicrobial component.

4. The disposable absorbent article of claim 3 in which the surfactant is selected from the group consisting of sulfate, sulfonate, phosphonate, phosphate, poloxamer, alkyl lactate, carboxylate, cationic surfactants, and combinations thereof.

5. The disposable absorbent article of claim 4 in which the surfactant is selected from (C₈-C₂₂) alkyl sulfate salts, di(C₈-C₁₈) sulfosuccinate salts, C₈-C₂₂ alkyl sarconsinate, and combinations thereof.

6. The disposable absorbent article of claim 1 wherein the disposable absorbent article comprises
a topsheet,
a backsheet joined to the topsheet, and
the fibrous absorbent material is disposed between the topsheet and the backsheet.

7. The disposable absorbent article of claim 1 wherein the degradable thermoplastic aliphatic polyester composition comprises a nonwoven.

8. The disposable absorbent article of claim 1 wherein the degradable thermoplastic aliphatic polyester composition comprises fibers or nanofibers.

9. The disposable absorbent article of claim 8 wherein the degradable thermoplastic aliphatic polyester composition fibers are distributed within the bulk of the absorbent material.

10. The disposable absorbent article of claim 1 wherein the disposable absorbent article is a tampon and the degradable thermoplastic aliphatic polyester composition is present in an amount sufficient to inhibit the production of TSST.

11. The disposable absorbent article of claim 9 wherein the disposable absorbent article is a tampon and the degradable thermoplastic aliphatic polyester composition is present in an amount sufficient to inhibit the production of TSST.

12. The disposable absorbent article of claim 1 wherein the degradable thermoplastic aliphatic polyester composition is present in an amount sufficient to inhibit the growth of Pseudomonas aeruginosa or Staphylococcus aureus.

13. The disposable absorbent article of claim 1 wherein the degradable thermoplastic aliphatic polyester composition is present in an amount sufficient to kill 99% of Pseudomonas aeruginosa or Staphylococcus aureus bacteria within a 3 hour period.

14. The disposable absorbent article of claim 1 wherein the disposable absorbent article is a woven, nonwoven, or knitted wipe formed at least in part of fibers formed from the degradable thermoplastic aliphatic polyester composition.

15. The disposable absorbent article of claim 1 wherein the disposable absorbent article is household wipe formed at least in part of fibers formed from the degradable thermoplastic aliphatic polyester composition.

16. A disposable absorbent article for absorbing body fluids comprising: an absorbent material and an at least one component formed at least in part from a degradable thermoplastic aliphatic polyester composition wherein the degradable thermoplastic aliphatic polyester composition comprises;
a) a thermoplastic aliphatic polyester;
b) an antimicrobial component incorporated therein wherein the antimicrobial component is a (C₇-C₁₄) saturated fatty acid monoesters of a polyhydric alcohol, and wherein the antimicrobial component is present in an amount greater than 1 percent by weight of the aliphatic polyester; and
c) an enhancer wherein the enhancer is either an alpha-hydroxy acid or an oligomer (that degrades to release an alpha-hydroxy acid) wherein the enhancer is present in an amount greater than 1 percent by weight of the aliphatic polyester.

17. The disposable absorbent article of claim 16 wherein the aliphatic polyester comprises polylactic acid, and wherein the antimicrobial component further comprises glyceryl monolaurate and/or propyleneglycol monolaurate, and wherein the enhancer further comprises an oligomer of lactic acid and glycolic acid.

18. A disposable absorbent article for absorbing body fluids comprising: an absorbent material and a degradable thermoplastic aliphatic polyester composition wherein the degradable thermoplastic aliphatic polyester composition comprises a) polylactic acid, b) glyceryl monolaurate and/or propyleneglycol monolaurate, and c) an oligomer of lactic acid and glycolic acid.

19. The disposable absorbent article of claim 1, wherein the aliphatic polyester is selected from the group consisting of poly(lactic acid), poly(glycolic acid), poly(lactic-co-glycolic acid), poly(3-hydroxybutyrate), blends, and copolymers thereof.

20. The disposable absorbent article of claim 19 in which the aliphatic polyester is semicrystalline.

21. The disposable absorbent article of claim 1 further comprising a plasticizer distinct from the antimicrobial component b) and enhancer c).

22. The disposable absorbent article of claim 1 in which the antimicrobial component is present in an amount greater than 5 percent by weight of the degradable thermoplastic aliphatic polyester composition.

23. The disposable absorbent article of claim 1 in which the antimicrobial component is present in an amount greater than 10 percent by weight of the degradable thermoplastic aliphatic polyester composition.

24. The disposable absorbent article of claim 1 in which the aliphatic polyester comprises at least 65 weight percent of the degradable thermoplastic aliphatic polyester composition.

25. The disposable absorbent article of claim 1 in which the antimicrobial component b) is selected from the group consisting of: (C₇-C₁₂) propylene glycol monoesters, glycerol monoesters, quaternary ammonium compounds and combinations thereof.

26. The disposable absorbent article of claim 1 in which the antimicrobial component b) is selected from the group consisting of propyleneglycol monolaurate, propyleneglycol monocaprylate, glycerol monolaurate, lauroylethylarginate, and combinations thereof.

27. The disposable absorbent article of claim 1 in which the enhancer is selected from the group consisting of benzoic acid, salicylic acid, mandelic acid, lactic acid, glycolic acid, glycolic acid oligomers, lactic acid oligomers glycolic/lactic acid copolymer oligomers, malic acid, adipic acid, succinic acid, sorbic acid, ethylenediaminetetraacetic acid and partial or fully neutralized salts thereof, butylatedhydroxytoluene, butylatedhydroxyanisole, methyl paraben, ethyl paraben, propyl paraben, butyl paraben, and combinations thereof.

28. The disposable absorbent article of claim 1 in which the enhancer is present in an amount ranging from greater than 0.1 to 20 percent by weight of the degradable thermoplastic aliphatic polyester composition.

29. A personal cosmetic or cleansing wipe comprising a fibrous absorbent formed at least in part from fibers of a degradable thermoplastic aliphatic polyester composition of:
a) a thermoplastic aliphatic polyester;
b) an antimicrobial component incorporated therein, selected from the group consisting of: (C₇-C₁₄) saturated fatty acid esters of a polyhydric alcohol, (C₇-C₂₂) unsaturated fatty acid esters of a polyhydric alcohol, (C₇-C₁₄) saturated fatty ethers of a polyhydric alcohol, (C₇-C₂₂) unsaturated fatty ethers of a polyhydric alcohol, (C₂-C₈) hydroxy acid esters of (C₇-C₂₂) alcohols, alkoxylated derivatives thereof, and combinations thereof, wherein the alkoxylated derivatives have less than 5 moles of alkoxide group per mole of polyhydric alcohol; with the proviso that for polyhydric alcohols other than sucrose, the esters comprise monoesters and the ethers comprise monoethers, and for sucrose the esters comprise monoesters, diesters, or combinations thereof, and the ethers comprise monoethers, diethers, or mixtures thereof, wherein the antimicrobial component is present in an amount greater than 1 percent by weight of the aliphatic polyester; and
c) an enhancer selected from the group consisting of alpha-hydroxy acids, beta-hydroxy acids, chelating agents, (C₂-C₆) saturated or unsaturated alkyl carboxylic acids, (C₆-C₁₆) aryl carboxylic acids, (C₆-C₁₆) aralkyl carboxylic acids, (C₆-C₁₂) alkaryl carboxylic acids, phenolic compounds, (C₁-C₁₀) alkyl alcohols, ether glycols, oligomers that degrade to release one of the aforesaid enhancers, and mixtures thereof in an amount greater than 0.1 percent by weight of the aliphatic polyester, except for phenolic compounds which are in an amount greater than 0.5 weight percent wherein the antimicrobial composition is formed by the antimicrobial component and enhancer.
